(19) United States
(12) Patent Application Publication
(10) Pub. No.: US 2025/0255847 A1
(43) Pub. Date: Aug. 14, 2025

(54) SIMMPYRAs: Novel Drugs for Triple Negative Breast Cancer

(71) Applicant: SIMMARON RSEARCH INC, Incline Village, NV (US)

(72) Inventors: Avik Roy, MILWAUKEE, WI (US); Alexander E. Arnold, MILWAUKEE, WI (US); Carl Gunnar Gottschalk, INDIANAPOLIS, IN (US)

(21) Appl. No.: 19/038,341

(22) Filed: Jan. 27, 2025

Related U.S. Application Data
(60) Provisional application No. 63/625,099, filed on Jan. 25, 2024.

(51) Int. Cl. A61K 31/381; A61P 35/00; C07D 333/64; G01N 33/50; G01N 33/68; G16B 15/30

(52) U.S. Cl. CPC A61K 31/381; A61P 35/00; C07D 333/64; G01N 33/5023; G01N 33/6872; G16B 15/30; G01N 2333/4703; G01N 2500/10

(57) ABSTRACT

The present invention relates to the discovery and characterization of novel, potent, and efficient STAT3 inhibitors which are two benzothiophene analogs named SIMMPYRA1 (SIM1) and SIMMPYRA2 (SIM2), and the method of their production by an in silico high throughput screening study. The presently disclosed STAT3 inhibitors specifically target STAT3 unregulated or irregular activation as in various STAT3-related diseases such as Triple-negative breast cancer (TNBC), a devastating form of breast cancer with poor life expectancy. The present invention discloses these inhibitors as potential prevention and treatment options for TNBC demonstrated by marked downregulation of several STAT3-dependent targets primarily IL6 and RANTES in TNBC cells, inducing apoptosis and/or downregulating cell growth suppression in multiple TNBC cells, and stimulating cytotoxicity and promoting tumor regression in vivo in a patient-derived xenograft (PDX) mouse model, without causing off-target side effects and preventing STAT3 activation-driven pathogenesis.

### PRIORITY

This application claims the benefit of priority to U.S. provisional application Ser. No. 63/625,099 filed on Jan. 25, 2024.

### FIELD OF THE INVENTION

The present invention generally relates to the discovery and development of inhibitors of Signal transducer and activator of transcription 3 (STAT3) and uses thereof. More specifically, the present invention relates to the discovery and development of novel small-molecule inhibitors of STAT3 for use in the prevention and treatment of STAT3-related diseases such as tumors, including Triple-Negative Breast Cancer (TNBC).

### SEQUENCE LISTING

The instant application contains a Sequence Listing submitted electronically in ASCII format and is hereby incorporated by reference in its entirety. Said ASCII copy, created on Apr. 27, 2025, is named TUP85565_19038341_seq.xml and is 59,26,912 bytes in size.

### INCORPORATION BY REFERENCE

All publications, patents, and patent applications mentioned in this specification are herein incorporated by reference in their entirety to the same extent as if each individual publication or patent application was specifically and individually indicated to be incorporated by reference.

### BACKGROUND OF THE INVENTION

Triple-Negative Breast Cancer (TNBC) is the most aggressive type of breast cancer [1]. Women of age less than 40 years are most susceptible to this tumor [2] with average life expectancy of no more than 5 years after diagnosis [3; 4]. Pathologically, triple-negative tumors are characterized with no expression of estrogen receptor (ER), progesterone receptor (PR), and extremely low expression of human epidermal growth factor receptor 2 (HER2/neu) [5]. Because of these mutations, TNBC cells are unresponsive to a class of compounds known as selective estrogen receptor modulators (SERM) [6] such as tamoxifen, toremifene, raloxifene, and ospemifene [7]. Therefore, until now there is a very limited therapy available that can prevent TNBC tumor progression and improve the poor prognosis of TNBC patients.

Activation of a nuclear protein named as Signal transducer and activator of transcription 3 (STAT3) [8] has frequently been found to be constitutively active in TNBC tumor tissue [9]. Upon activation, STAT3 enters the nucleus and binds to the promoter of tumor-promoting factors resulting in the uncontrolled growth of tumor cells [10]. Recently, applications of STAT3 inhibitors were introduced for the regression of different forms of cancers [11; 12; 13]. Some of these effective STAT3 inhibitors such as STX-0119 (lymphoma/glioblastoma) [14], LL1 (colon cancer) [15], S3I-201 (liver cancer) [16], CPA-7 (prostate cancer) [17], and stattic (TNBC) [18; 19; 20] effectively promote tumor regression at a concentration of micromolar range. For example, stattic, a potent STAT3 inhibitor (IC50=5 μM) effectively causes apoptosis of TNBC tumors [18] at 10-20 μM doses. Therefore, these compounds may be associated with off-target side effects. SD-36, another known STAT3 inhibitor was reported to effectively reduce tumor burden if administered at 25-50 mg/Kg body weight (bwt) dose [19]. At that high dose, SD-36 may also display off-target effects. Moreover, the degradation of STAT3 may cause the impairment of neurological functions including myelination, oligodendrocyte differentiation, and the induction of neuronal plasticity and cognition [21; 22]. Therefore, long-term treatment with protac inhibitor and the degradation of STAT3 may cause neurological issues. Hence, an effective STAT3 modulator is warranted to selectively inhibit the activation of STAT3 in tumor tissue, promote tumor apoptosis at a very low concentration (nanomolar or nM range), and display no off-target side effects.

Accordingly, there is a problem in the art and a need for an effective STAT3 modulator to selectively inhibit the activation of STAT3 in tumor tissue, promote tumor apoptosis and regression of TNBC at a very low concentration (nanomolar (nM) range), and display no off-target side effects. The present invention provides a solution to the aforesaid problem by providing two novel, potent and effective STAT3 inhibitors referred to as SIMMPYRA-1 (SIM1) and SIMMPYRA-2 (SIM2).

### SUMMARY OF THE INVENTION

The following listing of embodiments is a nonlimiting statement of various aspects of the invention. Other aspects and variations will be evident in light of the entire disclosure.

The present invention provides an in silico-techniques based discovery and development of two potent and effective small-molecule inhibitors of STAT3 referred to as SIMMPYRA-1 (SIM1 or SIM-1 used here interchangeably throughout) and SIMMPYRA-2 (SIM2 or SIM-2 used here interchangeably throughout), both with a chlorobenzothiophene backbone.

The present disclosure provides an effective small-molecule inhibitor of STAT3 referred to as SIM1 which has a molecular formula of C₁₃H₁₂ClNO₃S that is 4-[(5-chloro-1-benzothiophen-3-yl)methylamino]-4-oxobutanoic acid in IUPAC notation with simplified molecular-input line-entry system (SMILES) notation: C1=CC2=C(C═C1Cl)C(═CS2)CNC(═O)CCC(═O)O (See https://pubchem.ncbi.nlm.nih.gov/compound/2811468) having the following structure:

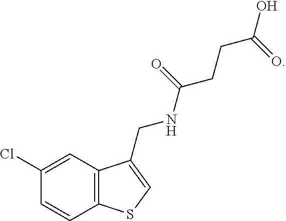 [SMILES: O=C(O)CCC(=O)NCc1csc2ccc(Cl)cc12]

The present invention also provides another effective small-molecule inhibitor of STAT3 referred to as SIM2 which has a molecular formula of C₁₄H₁₃ClN₂O₄S that is ethyl 2-acetamido-7-chloro-6-(hydroxyiminomethyl)-1-benzothiophene-3-carboxylate in IUPAC notation with SMILES notation: CCOC(═O)C1=C(SC2=C1C═CC(═C2Cl)C═NO)NC(═O)C (See https://pubchem.ncbi.nlm.nih.gov/compound/686160) having the following structure:

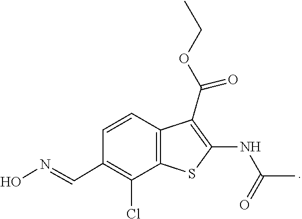 [SMILES: CCOC(=O)c1c(NC(C)=O)sc2c(Cl)c(/C=N/O)ccc12]

Further, the present invention provides detailed biochemical analyses revealing that both these compounds efficiently bind to STAT3 protein, particularly and selectively the activated STAT3 protein, inhibit phosphorylation at its critical tyrosine residue (Tyrosine 705 or Y705), suppress the expressions of STAT3-dependent tumor-promoting factors, and promote tumor apoptosis at nanomolar (nM) concentrations, making them more potent and advantageous over any other known STAT3 inhibitors. Thus, the present disclosure provides a new therapeutic option to treat TNBC and potentially other STAT3-dependent cancers.

An aspect of the present invention provides a compound having Formula (I), or an isomer, a structural analog, a chemical analog, or a pharmaceutically acceptable salt thereof:

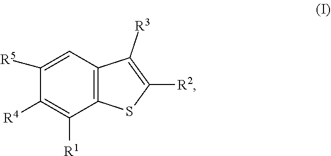

wherein R¹ is selected from a group consisting of —Cl, and —H,
wherein R² is selected from a group consisting of

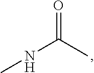

### and —H,

wherein R³ is selected from a group consisting of

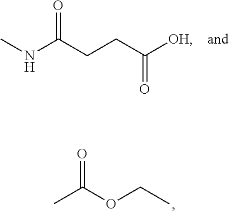

wherein R⁴ is selected from a group consisting of

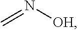

### and —H, and

wherein R⁵ is selected from a group consisting of —Cl, and —H.

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention, and are incorporated in and constitute a part of the present invention and, together with the description, serve to explain the principle of the invention.

In the drawings,

FIG. 1 illustrates an embodiment of the present invention and illustrates the chemical structures of all compounds selected in the in-silico method as disclosed in the present invention for screening against STAT3. Thirty-one compounds with molecular structure and Chemical Abstract Service (CAS) numbers have been listed and illustrated here. All these compounds have a benzothiophene backbone or equivalent structures.

FIG. 2 illustrates an embodiment of the present invention and illustrates the method of screening and synthesis of SIMMPYRA-1 (SIM-1) and SIMMPYRA-2 (SIM-2). (A) A total of 32 compounds were docked in the SH2 domain of STAT3 in the SwissDock server. The details of all screened compounds are shown in FIG. 1. The scatterplot summarizes the distributions of compounds combining full fitness energy (kCal/mol) versus free energy change (kCal/mol) of docking. The energy distributions of SIMMPYRA-1 (SIM-1) and SIMMPYRA-2 (SIM-2) are shown as 1 and 2 respectively in the plot. Chemical Formula of (B) SIM-1 and (C) SIM-2 are displayed with full fitness and free energies of docking. (D) The SH2 domain of STAT3 (PDB ID: 1BG1) shown by the arrowhead was targeted for docking STAT3 with SIM-1 and SIM-2. The most stable docking poses of (E) SIM-1 and (F) SIM-2 in the SH2 domain of STAT3 were built in Chimera (UCSF) software and then displayed. (G) The synthesis schema of SIM-1 was shown. (H) The LCMS characterization of SIM-1 showed a base peak at 298 (e/z). (I) Synthetic steps of SIM-2 were shown in a scheme with three labeled intermediates named IM1, IM2, and IM3.

FIG. 3 illustrates an embodiment of the present invention and illustrates ¹H and ¹³CNMR analyses of SIM-1.

FIG. 4 illustrates an embodiment of the present invention and illustrates ¹H and ¹³CNMR analyses of SIM-2.

FIG. 5 illustrates an embodiment of the present invention and illustrates evaluations of SIM-1 and SIM-2 as STAT3 inhibitors. (A) Protein thermal shift assay was performed to assess the melting of purified recombinant STAT3 (blue; 0.5 μg protein) (Tₘ=melting temperature=68.88° C.). The representative melting curves were derived after adding SIM-1 (red; 4 nM), SIM-2 (purple; 8 nM), and positive control stattic (grey; 20 nM) to the STAT3 protein. The differences of melting temperature (ΔTm) between pure protein and protein+ligands are −4.54° C., −4.16° C., and 0.39° C. for SIM-1, SIM-2, and stattic (ΔTm^{cut-off}>2° C.). (B) Phospho protein array analysis was conducted in cell lysates of MDA-MB-468 cells treated with DMSO control (top left), 25 ng/mL EGF (top right), EGF+10 nM SIM-1 (bottom left), and EGF+SIM-2 (bottom right) for 24 hrs. Each colored arrowhead represents a specific phosphoprotein, and the description was added in the bottom of array images. S=serine and Y=tyrosine. Relative levels of (C) Tyrosine 705 (Y705) phosphorylated STAT3, (D) Serine 727 (S727) phosphorylated STAT3, (E) Tyrosine 693 (Y693) phosphorylated STAT4, (F) Tyrosine 694 (Y705) phosphorylated STAT5A had been derived after normalizing the spot densities (n=5) with respective β-actin spots. Results are mean±SD of five different spot analyses. One-way ANOVA was adopted to test the significance of mean between groups at **p<0.01, and ****p<0.0001. Ns=no significance. Thermal shift assays of increasing doses of (G) SIM-1, (H) SIM-2, and (I) stattic with purified STAT1 protein (0.5 μg protein). The respective ΔTm values were given with the plot after matching with the color code. Similarly, full-length STAT5b protein melting was assessed with (J) SIM-1, (K) SIM-2, and (L) stattic. Results are confirmed after three different experiments.

FIG. 6 illustrates an embodiment of the present invention and illustrates dose-dependent curves of maximum fluorescence for SIM1 and SIM2. Protein thermal shift assay of recombinant STAT3 was performed with increasing doses of (A) SIM-1, (B) SIM-2, and (C) positive control “Stattic”. The maximum fluorescence for each dose was derived and then plotted in GraphPad Prism software with respective doses. The resultant non-liner hyperbolic curves provide half-maximal binding (EC50) for all three compounds.

FIG. 7 illustrates an embodiment of the present invention and illustrates in-silico binding analysis of SIM-1 with SH2 domain of STAT1, STAT2, and STAT5.

FIG. 8 illustrates an embodiment of the present invention and illustrates in-silico binding analysis of SIM-2 with SH2 domain of STAT1, STAT2, and STAT5.

FIG. 9 illustrates an embodiment of the present invention and illustrates promoter recruitment assay of STAT3 in IL6 promoter. (A) MDA-MB-468 cells were treated with 5, 10, and 20 ng/mL doses of EGF for 5 hrs followed by immunoblot (IB) analyses of Y705 phospho and S727 phospho STAT3 in nuclear extracts. Histone 3 (113) IB was run as control. (B) EMSA analyses were performed in nuclear extracts of EGF (20 ng/mL)- and EGF+SIM-1 (20 nM)-treated MDA-MB-468 cells against the STAT3-specific oligonucleotide probe (Li-Cor bioscience; Cat #P/N: 829-07922) or SEQ ID NO: 1 and resolved in 6% DNA retardation gel (Invitrogen). (C) Chromatin immunoprecipitation assay was performed in the nuclear extracts of MDA-MB-468 cells treated with EGF or EGF+SIM-1 for 5 hrs. Y705P STAT3 antibody was used for chromatin immunoprecipitation and IgG precipitation was performed as a control.

FIG. 10 illustrates an embodiment of the present invention and illustrates SIM-1 and SIM-2 inhibit expressions of IL6, RANTES, and other STAT3-dependent tumor promoting genes and proteins. (A) A gene array of STAT3 dependent 66 genes (listed in Table 1) was performed in mRNA samples of MDA-MB-468 cells. Realtime PCR plate coated with 66 primer sets of STAT3-dependent genes was run for four conditions including control, EGF (25 ng/mL), EGF+SIM-1 (10 nM) and EGF+SIM-2 (10 nM). The resultant Ct value was normalized with four different housekeeping genes, and then summarized with a heatmap analyses drawn in Morpheus server of Broad Institute. (B) Predictive gene to gene interaction analysis was done by STRING network analyses of downregulated genes in SIM-1- and SIM-2-treated MDA-MB-468 cells, which revealed that IL6 was in the primary node of gene interaction network. (C) The cloning of STAT3 response element of IL6 promoter upstream of GFP reporter was performed by GeneCopoea genome editing service (Rockville, MD). The bit score map and sequence of consensus STAT3 response element or SEQ ID NO: 2 was shown below. EGFP reporter assay was performed in EGF-induced (20 ng/mL) MDA-MB-468 after treatments of increasing doses of (D) SIM-1 and (E) SIM-2 for 5 hrs. Briefly, MDA-MB-468 cells were transfected with the plasmid for 24 hrs using Lipofectamine transfection strategy followed by the treatment with EGF (20 ng/mL) alone and together with increasing doses SIM-1 and SIM-2 for additional 5 hrs. After that live cells were recorded at Ex:Em=485/535 nm wavelength for GFP activity. (F) The scrambled STAT3 response element in IL6 promoter or SEQ ID NO: 3 was also synthesized partnering with GeneCopoea. One-way ANOVA (treatment as effector) was adopted to test the significance between groups at **p<0.01, #p<0.005, and *p<0.05. (G) The GFP reporter activity was measured where the scrambled gene was cloned upstream of eGFP reporter. Results are mean±SD of three different experiments Ns=no significance. (H) Realtime PCR analyses of IL6 was performed in MDA-MB-468 cells maintained under control, EGF (25 ng/mL), EGF+SIM-1(20 nM), and EGF+SIM-2 (20 nM) conditions for 5 hrs. (I) ELISA analysis of IL6 was performed after 48 hrs in MDA-MB-468 cells kept under similar treatment conditions. (J) Realtime PCR (5 hrs post-treatment) and (K) ELISA analyses of RANTES (48 hrs post treatment) were performed under similar condition. Results are mean±SD of three different experiments. One-way ANOVA (treatment as effector) was adopted to test the significance between groups at ***p<0.001, **p<0.01, and *p<0.05.

FIG. 11 illustrates an embodiment of the present invention and illustrates SIM-1 and SIM-2 induced apoptosis in MDA-MB-468 TNBC cells, but not HMEC control cells. MDA-MB-468 cells were treated with increasing doses of (A) SIM-1 and (B) SIM-2 for 48 hrs followed by lactate dehydrogenase (LDH) release assay in the supernatants. The result was offset with cell-bound LDH and presented as a percent of DMSO control as discussed under method section. Half-maximal cytotoxicity or CT50 analyses were performed plotting maximum response as an index of increasing doses of (C) SIM-1 and (D) SIM-2. AnnexinV (green) and propidium iodide (red) dual-labeling were performed to evaluate apoptotic response in MDA-MB-468 cells once treated with increasing doses of (E) SIM-1 and (F) SIM-2 for 24 hrs. Nuclei were stained with DAPI. TUNEL assay was performed in MDA-MB-468 cells after treatment with 25 nM of (G) SIM-1. Brown-colored TUNEL bodies were visualized at cresyl violet-stained blue nuclei. (H) The number of TUNEL bodies were counted as a percent of 100 cresyl violet-stained blue cells. Ten independent images were counted for the quantification study. Similarly, SIM-2 was assessed for (I) TUNEL and (J) subsequent quantification analyses. One-way ANOVA was performed to test the significance of mean between groups at **p<0.01 and ****p<0.0001. (K) LDH release assay was performed in non-tumor origin human mammary epithelial cells (HMEC) once treated with increasing doses of SIM-1 and SIM-2 for 48 hrs. Ns=no significance. (L) Annexin V (green) and propidium iodide (red) staining was performed in 25 nM SIM-1- and SIM-2-treated HMEC cells. Nuclei were stained with DAPI. Results are confirmed after three different experiments.

FIG. 12 illustrates an embodiment of the present invention and illustrates SIM-1 and SIM-2 arrest the growth of human TNBC tumor in patient-derived xenograft mouse model. TM00096 tumor (origin of invasive ductal carcinoma isolated from a 52-year-old white female) was seeded in the flank of 6-week-old female SCID mice by Jackson Laboratory and then shipped to our research facility. Once the tumor reached ˜4 mm in size (˜30 mm³ in volume), n=6 mice/group were treated by vehicle (5% DMSO), SIM-1 and SIM-2 (2 mg/Kg Bwt mixed with 5% DMSO) via i.p. for 2 weeks in every alternate day. The tumor size was monitored accordingly during the treatment and two more weeks after the last dose to evaluate the recurrence. After 4 weeks, Mice received IRDye 800-tagged 2DG (2-deoxy glucose) probe via tail vein and then imaged in infrared scanner. (A) Representative heatmap images for N=5 mice per group showed the tumor (red=high signal) in a blue background. Red spots enclosed in a squares are tumor tissue. Other red spots are non-specific organs with high metabolic activity. (B) Tumor size was measured every alternate day for 4 weeks by a digital caliper and then the volume was calculated using the formula 0.5*Length*Width². The representative scatter plot displayed a significant loss of growth in both SIM-1 (red) and SIM-2 (purple)-treated groups compared to the vehicle-treated group as shown for n=6 animals with mean±SEM. (C) The tumors harvested after 4 weeks were imaged with a scalebar and displayed for morphometric representation. (D) Body weight was recorded during the course of the study to monitor the adverse metabolic outcome due to the drug administration. (E) The heart rate was monitored at 0-, 7-, 14-, and 28-days post SIM-1 and SIM-2 treatments by MouseStat Jr console (Kent Scientific) as per the manufacturer's instruction. (F) At the end of the study, the gross motor performance was recorded for n=6 (i) vehicle, (ii) SIM-1-, and (iii) SIM-2-treated mice. Representative track plots were displayed to nullify the possibility of movement impairment due to the tumor growth. Gross movement parameters including (G) the total distance traveled, and (H) the average speed were monitored and plotted by scatter histograms (n=6/group; not significant as shown by one-way ANOVA).

FIG. 13 illustrates an embodiment of the present invention and illustrates the effects of SIM-1 and SIM-2 on the growth arrest of TNBC tumors. (A) H&E staining of tumor parenchyma displays morphometric properties of tumor cells in vehicle, SIM-1-, and SIM-2-treated tumors. hematoxylin-stained blue cells appeared to be larger and dense in vehicle-treated group and seemed to acquire normal size after SIM-1 and SIM-2 treatment. The higher magnification images were given below. (B) Area of 50 randomly selected cells (10 different images from n=5 mice/group) from three different groups were measured using polygon tool of ImageJ software and then the scatter histogram analysis was performed using GraphPad Prism 10 statistical software package. The significance of mean between groups (***p<0.001 versus control) was calculated by one-way ANOVA analysis (effector: treatment). (C) IHC staining of cell growth marker Ki-67 in vehicle-, SIM-1-, and SIM-2-treated tumors represents the cell growth property of tumor cells. (D) Scatter histogram analysis demonstrated the comparison of Ki67-ir cells among three groups. Both hematoxylin-ir nuclei (total cells) and Ki-67-ir cells were counted in n=15 images per group, converted in percent scale, and then plotted as scatter histogram. ****p<0.0001 vs. control as derived by one-way ANOVA. (E) IHC staining of cell proliferation marker PCNA in three different groups. (F) Scatter histogram analysis of Ki-67-ir cells counted from n=15 images per group and then plotted as percent scale. Significance of mean between groups was tested by One-way ANOVA that resulted in ****p<0.0001 vs. control. (G) A gene array of 84 genes in a validated human breast cancer gene array (RT² Profiler™ PCR array; Cat #PAHS-131ZA; Qiagen) was conducted in cDNA samples extracted from TNBC tumors of vehicle, SIM-1- and SIM-2-treated (n=3) groups. The resultant gene expressions were converted in log scale and then compared with the vehicle in (G) SIM-1 and (H) SIM-2 treated groups as shown in scatter plots. The upregulated (red dots) and the downregulated (green dots) genes were labeled. Upregulated genes such as CDKN1A (Cyclin Dependent Kinase Inhibitor 1A) and CDKN2A (Cyclin Dependent Kinase Inhibitor 1A) inhibit cell division. Downregulated genes such as CCND1 (Cyclin D1), CCND2 (Cyclin D2), CCNE1 (Cyclin E1), are CDK2 (Cyclin-dependent kinase2) stimulated cell division. One of the downregulated genes MKI67 encodes for Ki-67 protein, which was also shown to be downregulated by immunostaining. (I) Validation of gene expressions of CCND1, CCND2, CCNE1, CDK2 genes were performed by real-time PCR analysis. (J) Upregulations of CDKN1A, and CDKN2A in SIM-1- and SIM-2-treated groups were validated by real-time PCR analyses. were Significance of mean calculated by one-way ANOVA resulted ***p<0.005 and ****p<0.001 versus respective vehicle-groups. The results are confirmed after three different experiments.

FIG. 14 illustrates an embodiment of the present invention and illustrates SIM1 and SIM2 treatment weakly induced apoptosis in TNBC tumor. (A) TUNEL staining to monitor apoptosis of tumor cells among all groups followed by (B) counting of TUNEL-ir cells from n=15 images per group and then plotting a scatter histogram analysis. ****p<0.0001 vs. control as derived by one-way ANOVA. (C) A protein array of 35 different apoptosis-related proteins (Human apoptosis array; Cat #ARY009; R&D Systems) was conducted in the tumor tissue extracts of three different groups. Red, purple, and green squares represent the expressions of Bad, Bax, and Cytochrome C proteins respectively. Black enclosures represent the positive controls. (D) Heatmap analysis summarizes the expressions of breast cancer 84 validated breast cancer-associated genes (RT² Profiler™ PCR array; Cat #PAHS-131ZA; Qiagen). These genes critically regulate the growth and apoptosis of breast cancer cells. The details of the genes can be found here (geneglobe.qiagen.com/us/product-groups/rt2-profiler-pcr-arravs/PAHS-131Z). Scatter plot analyses in FIG. 13G and FIG. 13H was performed to compare the relative expressions of apoptotic genes and growth suppressive genes between the vehicle- and SIM-1-treated as well as the vehicle- and SIM-2-treated groups.

FIG. 15 illustrates an embodiment of the present invention and illustrates SIM 1 and 2 inhibited Y705P phosphorylation of STAT3 and the expressions of STAT3-dependent tumor supportive genes. (A) DAB immunostaining of Y705P STAT3 in tumor parenchyma of all three groups. (B) Quantitative analysis of Y705P STAT3-ir cells was performed by counting Y705P STAT3 cells in 15 different images, converting into a percent scale, and then plotting in a scatter histogram. On-way ANOVA followed by multiple comparison test indicated ****p<0.0001 versus control. (C) DAB immunostaining of S727P STAT3 followed by a (D) quantitative analysis of S727P STAT3-ir cells was performed in 15 different images and then plotted as a percent of total cells by a scatter histogram. On-way ANOVA represents no significance (NS) between groups. (E) Immunoblot analyses of Y705PSTAT3 (Rabbit 10 antibody Cat #710093; Invitrogen), S727PSTAT3 (Mouse 1° antibody cat #MA5-15208; Invitrogen) followed by normalization with loading control β-actin (Mouse 1° antibody cat #MA1-744; Invitrogen). (F) A cDNA-based gene array of 66 STAT3-dependent tumor promoting genes were analyzed in TNBC tumors of vehicle-, SIM-1-, and SIM-2-treated mice. (G) A scatter plot summarized the gene array results. Based on the relative levels of expressions, gene levels were classified as low, moderate, and highly expressed. Results are confirmed after three different experiments.

### DETAILED DESCRIPTION OF THE INVENTION

Detailed embodiments of the present invention are disclosed herein. However, it is to be understood that the disclosed embodiments are merely exemplary of the present invention, which may be embodied in various systems. Therefore, specific details disclosed herein are not to be interpreted as limiting, but rather as a basis for teaching one skilled in the art to variously practice the present invention.

All illustrations of the drawings are for the purpose of describing selected versions of the present invention and are not intended to limit the scope of the present invention.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a microbe, a microbial formulation, a pharmaceutical composition, and/or a method that “comprises” a list of elements (e.g., components, features, or steps) is not necessarily limited to only those elements (or components or steps), but may include other elements (or components or steps) not expressly listed or inherent to the microbe, microbial formulation, pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

The terms “subject,” “patient” and “individual” as used herein are used interchangeably herein to refer to a vertebrate, including mammals and humans. A “subject,” “patient” or “individual” as used herein, includes any animal that exhibits pain that can be treated with the compositions, formulations or systems, and methods contemplated herein, and that includes laboratory animals, farm animals, and domestic animals or pets, non-human primates, and human are included.

As used herein, the term “amount” refers to “an amount effective” or “an effective amount” of a cell to achieve a beneficial or desired prophylactic or therapeutic result, including clinical results.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g., a symptom associated with the disease or condition being treated. As such, “treatment” also includes situations where the disease, disorder, or pathological condition, or at least symptoms associated therewith, are completely inhibited (e.g., prevented from happening) or stopped (e.g., terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, the term “STAT3 inhibitor” refers to substances that target signal transducer and activator of transcription 3 (STAT3) proteins, which belong to a family of cytoplasmic transcription factors. These STAT3 inhibitors include small molecule inhibitors, natural product inhibitors, peptides/peptidomimetics, relevant analogs, as well as their core framework.

As used herein, the term “STAT3-related diseases” refers to diseases and disorders involving unregulated or irregular activation of STAT3 that results in inhibition of immune mediators and promotion of immunosuppressive factors, including cancers, autoimmune diseases, COVID-19, triple-negative breast cancer (TNBC), and myalgic encephalomyelitis or chronic fatigue syndrome (ME/CFS).

As discussed above, there remains a need in the art for effective STAT3 inhibitors that are more potent and produce less off-target effects for use in the prevention and/or treatment of STAT3-related diseases including triple-negative breast cancer (TNBC).

An embodiment of the present invention provides a STAT3 inhibitor, wherein the STAT3 inhibitor comprises a chlorobenzothiophene backbone.

An embodiment of the present invention provides a compound having Formula (I), or an isomer, a structural analog, a chemical analog, or a pharmaceutically acceptable salt thereof:

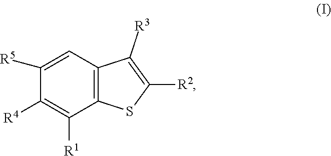

wherein R³ is selected from a group consisting of —Cl, and —H,
wherein R² is selected from a group consisting of

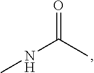

### and —H,

wherein R³ is selected from a group consisting of

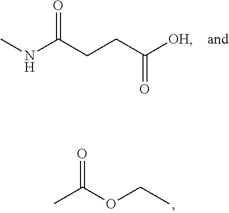

wherein R⁴ is selected from a group consisting of

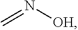

### and —H, and

wherein R⁵ is selected from a group consisting of —Cl, and —H.

Another embodiment of the present invention provides the compound as disclosed hereinabove, wherein the compound has Formula (II), or an isomer, a structural analog, a chemical analog, or a pharmaceutically acceptable salt thereof:

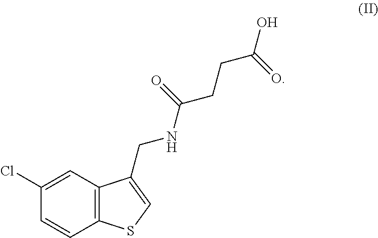 [SMILES: O=C(O)CCC(=O)NCc1csc2ccc(Cl)cc12]

Another embodiment of the present invention provides the compound as disclosed hereinabove, wherein has Formula (III), or an isomer, a structural analog, a chemical analog, or a pharmaceutically acceptable salt thereof:

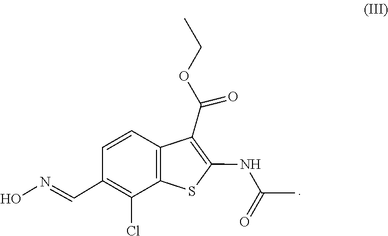 [SMILES: CCOC(=O)c1c(NC(C)=O)sc2c(Cl)c(/C=N/O)ccc12]

Another embodiment of the present invention provides the compound as disclosed hereinabove, wherein the compound is a small-molecule inhibitor targeting signal transducer and activator of transcription 3 (STAT3) that inhibits only STAT3 at a nanomolar concentration not other isoforms of signal transducer and activator of transcription factors (STATs), and wherein the compound stimulates cytotoxicity and induces apoptosis of cells with irregular STAT3 activation at nanomolar concentration, does not cause off-target side effects, and prevents STAT3 activation-driven pathogenesis.

Another embodiment of the present invention provides the compound as disclosed hereinabove, wherein the compound is comprised in a pharmaceutical composition, wherein the pharmaceutical composition comprises the compound of claim 1; and at least one of pharmaceutically acceptable carriers; excipients; diluents; adjuvants; and vehicles, wherein the pharmaceutical composition further comprises an additional therapeutic agent, wherein the additional therapeutic agent is a chemotherapeutic drug, an antiproliferative agent, an immunosuppressor, an immunologic stimulant, an anti-inflammatory reagent, or a combination thereof, wherein the compound is a small-molecule inhibitor targeting signal transducer and activator of transcription 3 (STAT3) that inhibits only STAT3 at a nanomolar concentration not other isoforms of signal transducer and activator of transcription factors (STATs), and wherein the compound stimulates cytotoxicity and induces apoptosis of cells with irregular STAT3 activation at nanomolar concentration, does not cause off-target side effects, and prevents STAT3 activation-driven pathogenesis.

An embodiment of the present invention provides a method of obtaining a compound of claim 1, the method comprising the steps of: (a) preparing a series of structurally related compounds having a benzothiophene backbone by extracting liner sequence of each compound in simplified molecular-input line-entry system (SMILES) format, and then converting them to three-dimensional Mol2 format with the help of Python-based algorithm; (b) screening the compounds of step (a) for their role as small-molecule inhibitors of signal transducer and activator of transcription 3 (STAT3) by employing an in-silico technique to assess binding of said compounds in the ligand binding pocket of STAT3 protein based on their docking with STAT3 in Swiss-Dock server; (c) identifying the most potent and efficient inhibitors of STAT3 from amongst the compounds of step (b) based on free-energy change and full-fitness energy ranking to identify the compound as disclosed herein; (d) synthesizing, and purifying the identified compound as disclosed herein; (e) structurally evaluating the synthesized and purified compound of step (d) by LCMS and NMR spectroscopy; (f) assessing the compound of step (e) by biochemical and cellular assays for selective binding and molecular interaction with STAT3 at a nanomolar concentration; (g) validating functional and selective inhibition of STAT3 activation at a nanomolar concentration not other isoforms of signal transducer and activator of transcription factors (STATs) in cell-based assays by the compound of step (f), wherein steps (a) to (g) result in obtaining the compound as disclosed herein as a potent and efficient selective inhibitor of STAT3 at a nanomolar concentration.

Another embodiment of the present invention provides the method as disclosed hereinabove, wherein the biochemical and cellular assays include protein thermal shift (PTS) assay, fluorescence polarization analyses, high-throughput STAT binding protein array assay, immunoblot assay, 6× luciferase reporter gene assay array for selective STAT3 selective binding and molecular interaction assessment at a nanomolar concentration of the compound as disclosed herein.

Another embodiment of the present invention provides the method as disclosed hereinabove, wherein the cell-based assays include assessment of specific STAT3 activation inhibition targeting suppression of phosphorylation at tyrosine 705 (Y705) of STAT3 protein, Lactate dehydrogenase cell cytotoxicity assay, dose-response analysis for apoptosis in cell-based assays, annexin-V/propidium iodide dual labeling assay, TUNEL staining assay, and apoptotic gene array assay at a nanomolar concentration of the compound as disclosed herein.

An embodiment of the present invention provides a method of preventing or treating a disorder or disease in a subject, the method comprising the steps of: (i) obtaining the compound as disclosed herein; (ii) preparing a pharmaceutical composition comprising the compound as disclosed herein; (iii) identifying the subject for administering the pharmaceutical composition comprising the compound as disclosed herein; (iv) assessing the subject for pre-administration vital signs and collecting biological samples from the subject to establish and record baseline physiological, metabolic profile, and medical history of the subject for pre-administration assessment; (v) administering a therapeutically effective amount of the pharmaceutical composition comprising the compound as disclosed herein to the subject; (vi) repeating the administration in step (v) as per an established protocol for treating the disorder or disease in the subject; (vii) collecting biological samples and recording the vital signs from the subject to establish and record baseline physiological, metabolic profile at each step post-administration of step (v) and step (vi) as per the established protocol for post-administration assessment; (viii) comparing the results obtained from the pre-administration assessment and post-administration assessment to check for the potency and efficiency of the compound as disclosed herein in preventing or treating the disorder or disease in the subject, wherein the disorder or disease is a signal transducer and activator of transcription 3 (STAT3) activation-dependent disorder or disease involving irregular activation of STAT3 protein, wherein the pre-administration and post-administration assessment include in-vivo measures based baseline assessment, and in-vitro assays conducted as biochemical and cellular assays, and cell-based assays on the biological samples obtained in steps (iv) and (vii), and wherein the compound as disclosed herein is a small-molecule inhibitor targeting signal transducer and activator of transcription 3 (STAT3) as a potent and efficient selective inhibitor of STAT3 at a nanomolar concentration.

Another embodiment of the present invention provides the method as disclosed hereinabove, wherein the disorder or disease includes triple-negative breast cancer (TNBC), other cancers with STAT3 activation including prostate cancer, liver cancer, leukemia, lymphoma, multiple myeloma, colorectal cancer, lung cancer, and brain cancers including glioblastoma, and neuroblastoma, different metabolic diseases including Coronavirus disease 2019 (COVID-19), myalgic encephalomyelitis or chronic fatigue syndrome (ME/CFS).

Another embodiment of the present invention provides the method as disclosed hereinabove, wherein the compound targets suppression of phosphorylation at tyrosine 705 (Y705) of STAT3 protein.

Another embodiment of the present invention provides the method as disclosed hereinabove, wherein the baseline assessment includes comprehensive documentation of patient characteristics including demographic information comprising data on age, gender, ethnicity; social factors including support system, employment status, lifestyle habits; medical history including existing medical conditions and/or comorbidities, severity of the primary disease; clinical symptoms including pain level, functional ability, fatigue levels, mood; routine and disease or disorder specific laboratory test values including disease or disorder specific blood tests and imaging results indicating disease progression and grade; quality of life measures including self-reported assessments of physical and mental well-being; and psychological factors including anxiety levels, depression, coping mechanisms.

Another embodiment of the present invention provides the method as disclosed hereinabove, wherein the biochemical and cellular assays include protein thermal shift (PTS) assay, fluorescence polarization analyses, high-throughput STAT binding protein array assay, immunoblot assay, 6× luciferase reporter gene assay array for selective STAT3 selective binding and molecular interaction assessment at a nanomolar concentration of the compound as disclosed herein.

Another embodiment of the present invention provides the method as disclosed hereinabove, wherein the cell-based assays include assessment of specific STAT3 activation inhibition targeting suppression of phosphorylation at tyrosine 705 (Y705) of STAT3 protein, Lactate dehydrogenase cell cytotoxicity assay, dose-response analysis for apoptosis in cell-based assays, annexin-V/propidium iodide dual labeling assay, TUNEL staining assay, and apoptotic gene array assay at a nanomolar concentration of the compound as disclosed herein.

Cell lines, Reagents, Antibodies, reporter clones, and Kits. MDA-MB-468 cells (Cat #HTB-132), Human mammary epithelial cells (Cat #PCS-600-010), HEK293T cells (Cat #CRL3216), HMEC growth factor cocktail (Cat #PCS-600-040), and HMEC media (Cat #PCS-600-030) were purchased from ATCC (American Type Culture Collection). Phospho Y705STAT3 antibody (Cat #MA5-41192), phospho S727STAT3 antibody (Cat #PSTAT3-340AP), beta-actin (Cat #MA1-140) antibodies, CyQUANT™ LDH Cytotoxicity Assay (Cat #C20301), Annexin V/Propidium iodide (PI) apoptosis detection kit (Cat #V13241), and protein thermal shift assay kit (Cat #4462263) were purchased from Thermo fisher. JAK/STAT Phospho antibody array membranes (Cat #PJS042) and reagent kit (Cat #KAS02) were purchased from Full moon Biosystems INC, DeadEnd™ Colorimetric TUNEL assay kit (Cat #G7360) was purchased from Promega. GFP reporter constructs with wild-type and scrambled STAT3 response elements of IL6 promoter were custom cloned in pEZX-PF02 plasmid vector and purchased from Genecopeia. STAT3 oligonucleotide probe (Cat #829-07922) or SEQ ID NO: 1 and IRDye680-labeled streptavidin (Cat #926-68079) were purchased from Li-cor Bioscience. Recombinant STAT3 protein (Cat #81095) was purchased from Active motif.

Synthesis of SIM1 and SIM2. Synthesis of SIM1 and SIM2 were designed and conducted by Milwaukee Institute for Drug Discovery, Milwaukee, WI, USA. It is described hereinbelow in Example 1 as a representative example of the disclosure of the present invention and should be read and understood along with it.

Synthesis of SIM1. Under Nitrogen, (118 mg, 1.0 mmol) succinic acid, (379 mg, 1.0 mmol)O-(Benzotriazol-1-yl)-N,N,N′,N′-tetramethyluronium hexafluorophosphate (HBTU), and (0.202 mL, 2.0 mmol) N-methylmorpholine (NMM) were dissolved in 5 mL of dry acetonitrile, the reaction mixture was then stirred at RT for 10 minutes. 197 mg, 1.0 mmol of 5-Chloro-1-benzothien-3-yl)methanamine was added, and the reaction mixture was then stirred at RT for hrs. The progress of the reaction was then checked by TLC (70% ethyl acetate: hexane), and then concentrated. The resulting residue was then dissolved in 50 mL chloroform and washed once with 50 mL of 1.0 M ammonium chloride solution and with 50 mL brine solution, then dried with magnesium sulfate and concentrated. The resulting residue was then purified using column chromatography, the following parameters were used: 90%:10% Methanol for 20 Column Volumes.

Synthesis of SIM2. The entire synthesis has 3 stages.

Stage 1: Synthesis of ethyl 2-acetamido-7-chloro-6-formyl-4,5-dihydrobenzo[b]thiophene-3-carboxylate. The Vilsmeier reagent was prepared by adding Phosphorous Oxychloride (2.3 mmol, 461 μL) dropwise to ice-cold dry DMF (2 mmol, 138 μL). The reaction mixture was then stirred at 0° C. for 10 minutes followed by 30 minutes of stirring at room temperature. To this mixture was added a solution of (1 mmol, 281 mg) of ethyl2-acetamido-7-oxo-4,5,6,7-tetrahydrobenzo[b]thiophene-3-carboxylate in 5 ml of dry dichloroethane. The reaction mixture was then stirred at RT for 30 minutes and then refluxed at 75° C. for 40 minutes. The progress of the reaction was then checked by TLC (100% ethyl acetate). The reaction mixture was then dissolved in 25 mL of dichloroethane, washed once with 50 mL of DI Water, 50 mL of 5% sodium bicarbonate, 50 mL of 10% sodium bicarbonate, and 50 mL of brine solution. The organic layer was then dried with magnesium sulfate and concentrated to yield a yellow powder. Recrystallization is not necessary. The reported yield was 138 mg or 42.2%.

Stage 2: Synthesis of ethyl 2-acetamido-7-chloro-6-formylbenzo[b]thiophene-3-carboxylate. 81 mg, 1 mmol of ethyl 2-acetamido-7-chloro-6-formyl-4,5-dihydrobenzo[b]thiophene-3-carboxylate and (0.342 mL, 1.4 mmol) of sulfuryl chloride in DCM solution were stirred in 3 mL of dry chloroform at RT for 30 minutes and then refluxed at 65° C. for 1.5 hours. The reaction progress was checked by mass spectrometry. The reaction mixture was then cooled, washed with 50 mL of DI Water, and then the organic layer was dried with magnesium sulfate and then concentrated to yield an off-white powder. The reported yield was 75 mg or 93.2%.

Stage 3: Synthesis of ethyl (E)-2-acetamido-7-chloro-6-((hydroxyimino)methyl)benzo[b]thiophene-3-carboxylate. 85 mg of ethyl 2-acetamido-7-chloro-6-formylbenzo[b]thiophene-3-carboxylate was dissolved in 3.5 mL of pyridine. 21 mg of NH2OH·HCl was then added. Stirred at RT for 24 hr. Checked TLC (50% EtoAc:Hex). Poured into DI water dropwise forming a precipitate. Filtered the precipitate for 24 hours after washing with water. The reported yield was 38 mg or 43% of white powder.

Gene array. Briefly, MDB-MB-468 cells were grown in 6 well plate. When 75% confluency was reached, cells were treated with SIM1 and SIM2 under serum-free condition for 5 hrs as described earlier [23]. After that, mRNA was isolated using the Gene Jet RNA purification kit (Cat. #K0732), quantified by Bio-Rad Smart Spec™ plus spectrophotometer, converted to cDNA by GoScript™ CDNA synthesis kit, as per the manufacturer's instructions. Real-time PCR array was performed with cDNA, forward and reverse primers, and SYBR green master mix following a two-stage cycle with melting in ABI7300 realtime PCR machine. The resultant Ct value was normalized by the average value of 4 housekeeping genes, converted in a log scale, and then plotted as a heatmap in Morpheus webserver of Broad Institute. The dendrogram represents the Euclidean distance of genes based on the expression level. Primers of 66 genes and 4 housekeeping genes were designed using primer select tool of NCBI website (https://www.ncbi.nlm.nih.gov/nuccore).

Protein array. The JAK/STAT phospho antibody array (Cat #PJSO42) was developed by Full Moon Biosystems, Inc. (Sunnyvale, CA). Each array membrane contains 42 highly specific antibodies of JAK/STAT pathway replicated five times in a row, embedded in a glass slide. Positive, negative, and housekeeping controls (beta-actin) were embedded along with following phospho-specific antibodies. JAK1 (Tyr1022), JAK2 (Tyr1007), JAK2 (Tyr221), MEK1 (Ser217), MEK1 (Ser221), MEK1 (Thr291), MEK2 (Thr394), p44/42 MAPK (Thr202), p44/42 MAPK (Tyr204), Raf1 (Ser259), Raf1 (Ser338), STAT1 (Ser727), STAT1 (Tyr701), STAT3 (Ser727), STAT3 (Tyr705), STAT4 (Tyr693), STAT5A (Ser780), STAT5A (Tyr694), STAT6 (Thr645), STAT6 (Tyr641), TYK2 (Tyr1054). Briefly, MDA-MB-468 cells were treated with 25 ng of EGF alone or either with 10 nM SIM1 or SIM2 for 6 hrs. After that, cells were scrapped with 1×TBS, lysed with lysis buffer supplemented with protease and phosphatase inhibitor cocktail, measured for total protein concentration (˜200 mg/mL), diluted 5 times with assay diluent buffer and then applied along with biotin-labeled probes on the glass membrane coated with antibodies. Glass membrane was soaked with 1× blocking buffer prior to the application of cell lysate. After applying cell lysate and biotin probe, the array slide was incubated at 4° C. for overnight, washed 4-5 times with 1× wash buffer, incubated with IRDye680-conjugated streptavidin, and incubated for another 2 hrs at room temperature. After that, slides were washed 4-5 times with 1× wash buffer, dried, and then imaged in Li-Cor Odyssey Sa instrument at 200 μm resolution and 7 focus offset.

LDH Assay. LDH Assay was performed as per manufacturer's instructions (Cat #: 2570393; Vendor: Thermo Fisher Scientific). MDA-MB-468 and HMEC cells were treated with SIMPYRA-1 and SIMPYRA-2 at doses of 2 nM, 5 nM, nM, and 25 nM. Each dose was measured in triplicates. After 48 hours of incubation at 37° C., 50 μL of soup from each well was harvested and transferred into a new 96 well plate. Each well was treated with 50 μL of reaction mix (Substrate Mixture+deionized water+Assay Buffer). The reaction was stopped with 50 μL of Stop Solution. The plate was read at 490 nm (background at 680 nm).

TUNEL Assay Protocol. TUNEL assay was performed as per manufacturer's instructions (Cat #: 0000525788; Vendor: Promega). Briefly, slides with MDA-MB-468 cells were treated with SIMPYRA-1 at doses of 25, 50, and 75 nM and SIMPYRA-2 at doses of 25, 50, 75, and 150 nM and incubated at 37° C. for 24 hours. Cells were washed with 1×PBS, equilibrated with Equilibration Buffer, and treated with TdT reaction mix (TdT Equilibration Buffer+Biotinylated Nucleotide Mix+TdT enzyme). Reaction was stopped using 2×SSC. Cells were incubated with Streptavidin HRP (diluted 1:500 in 1×PBS), washed with 1×PBS, and stained with DAB Solution (DAB Substrate 1× Buffer+10× Chromogen).

Annexin V/PI Dual Staining Protocol. Annexin V/PI Dual Staining was performed as per manufacturer's instruction (Cat #: 2752665; Vendor: Thermo Fisher Scientific). Chamber slides of MDA-MB-468 cells in serum-free media were treated with SIMPYRA-1 and SIMPYRA-2 at doses of 2 nM, 5 nM, 10 nM, nM, and 50 nM for 24 hours at 37° C. Each well was washed with 1×PBS, then treated with 1× annexin-binding buffer, 100 ug/mL PI solution, and Annexin V solution. Chamber slides were incubated in the dark at room temperature for 2 hours. Each well was washed twice with 1×PBS and once with 1:500 dilution of DAPI in 1×PBS. Cells were imaged using CaptaVision+™ Software.

Promoter analysis and Chromatin Immunoprecipitation (ChIP) assay. Promoter sequences of IL6 were extracted from chromosome #7 (PubMed.gov Seq. ID: NG_011640.1). After that, with the help of MatInspector (Genomatix software suite), these sequences were analyzed for STAT3 binding sites, resultant responsive elements of STAT3 protein were identified with matrix match score>0.9, and then displayed with bit score diagram. Next, primer sequences were designed from 125-150 bp of nucleotides around the response element. Primer designing was performed with the help of the “primer blast” module of PubMed (Link: https://www.ncbi.nlm.nih.gov/tools/primer-blast/). Primers used to amplify sonicated DNA are as follows.

IL6 promoter (product length=145 bp):

Sense:
5′AGACCTGGTTTGGGGATCTTAAT 3′
or
SEQ ID NO: 4;
Antisense:
5′AGCAATGAGCTGGTCTCCCA 3′
or
SEQ ID NO: 5.

Recruitment of STAT3 to the IL6 promoter had been analyzed in HEK293T cells by ChIP in vitro as described previously. Briefly, cells were treated with 25 ng/mL EGF and EGF+SIM1(5 nM) for 5 hrs under serum-free condition, fixed with 4% paraformaldehyde, quenched with 5M glycine, washed, extracted for nuclear fraction (Abcam nuclear extraction kit; Cat #ab113474), sonicated (Swift lab ultrasonic processor; pulse sonication at 25 KHz) in Tris-EDTA buffer (pH 7.6), incubated with antibodies at rotating condition overnight followed by pulling down with protein A agarose, dissociated the DNA-Protein complex with Chelex® 100 (Sigma), centrifuged, and then performed realtime PCR with aforementioned primers via SYBR Green detection method.

ELISA assay. IL6 (BD OptEIA™; Cat #555220) and RANTES (Thermo; Cat #EHRNTS) ELISA was performed as per manufacturer's protocol. Briefly, a 96-well plate was coated with primary antibody (or precoated plate), followed by incubation with supernatants for 24 hrs at 4° C., washed with 1×TBS (or 1× Wash buffer) for 3 times, incubated with biotin-conjugated secondary antibody, then with HRP-conjugated antibody, and finally developed with TMB substrate.

Electrophoretic Mobility Shift Assay (EMSA). EMSA was performed in HEK293T cells as described before. Mouse primary microglial cells were treated with 25 ng/mL EGF alone or together with SIM1 (5 nM) for 5 hrs followed by nuclear fractionation with nuclear extraction kit (Abcam). After that, the reaction was performed with 2 μL 10× Binding Buffer (100 mM Tris, 500 mM KCl, 10 mM DTT; pH 7.5)+1 μL of Poly(dI·dC) 1 μg/μL in 10 mM Tris, 1 mM EDTA; pH 7.5+2 μL of 25 mM DTT/2.5% Tween®20+10 μL of Water+1 μL of IRDye 700 NFκB+2 μL of nuclear extract (5 μg) (=total 20 μL reaction). During that reaction, 6% DNA gel was pre-run at 0.5% TBE for 30 minutes (120 V) and then the reaction mixture was loaded and electrophoresed for 90 mins at 90 V. The gel was carefully transferred in a glass tray and imaged at Li-Cor Odyssey Sa imager at 200 μm resolution.

Immunoblot assay. Immunoblot assay was performed as described before [24]. Briefly, cell lysates were mixed with 5× Lamelli buffer, electrophoresed in 4-12% Tris-Glycine gel in tris-glycine SDS running buffer, transferred in a nitrocellulose membrane (P/N 926-31090; Li-Cor Biosciences, Lincoln, NE) in tris-glycine transfer buffer, incubated with appropriate primary antibodies overnight under shaking condition, labeled with IRDye700 and 800-tagged secondary antibodies for 2 hrs at room temperature in an orbital shaker, and then imaged in Odyssey Sa imager at 200 μm resolution.

Immunohistochemical (IHC) analyses. Tumor tissues were embedded in paraffin, cut at a thickness of 5 μm in Leica microtome, attached to a charged slide, dried in a dry bath at 37° C. for at least 48 hrs., and then progressed for IHC analysis. The IHC analysis was performed where, briefly, sections were deparaffinized by xylene and rehydrated by ethanol gradient (100%>70%>50%>20%; v/v) followed by complete rehydration by water. After that sections were blocked by Intercept® (TBS) Blocking Buffer (Licor Biosciences, Lincoln, NE) for 30 minutes, incubated with primary (1°) antibodies (Refer to Table 1) overnight at 4° C., washed with 1×TBST wash buffer for 3 times, incubated with biotinylated anti-mouse (1:200 dilution) or anti-rabbit (1:200 dilution) secondary (2°) antibodies (ThermoFisher), for 2 hrs., reacted with VECTASTAIN® Elite® ABC-HRP Kit, Peroxidase (Cat #PK-6100; Vector Laboratories) for 1 hr. as per manufacturer's recommendation, and finally stained with DAB chromogenic solution (Cat #SK-4100; Vector Laboratories).

TABLE 1
Primary (1°) antibodies

| Antibody | Cat# | Vendor | Host | Application |
|---|---|---|---|---|
| Anti- | MA5-41192 | Invitrogen | Rabbit | IB: 1:500; |
| Y705PSTAT3 | | | | IHC: 1:100 |
| Anti- | 44-384G | Invitrogen | Rabbit | IHC: 1:100 |
| S727PSTAT3 | | | | |
| Anti-Ki67 | 27309-1-AP | Protein Tech | Rabbit | IHC: 1:500 |
| Anti-PCNA | 24036-1-AP | Protein Tech | Rabbit | IHC: 1:500 |
| Anti-β actin | PA5-85271 | Invitrogen | Rabbit | WB: 1:500 |
| Anti-H3 | 29200-1-AP | Invitrogen | Rabbit | WB: 1:500 |
| Anti-HA | 51064-2-AP | Protein Tech | Rabbit | WB: 1:500 |
| Anti-FLAG | 66008-4-Ig | Protein Tech | Mouse | WB: 1:500 |
| Anti- | PSTAT4-440AP | FabGennix | Rabbit | WB: 1:500 |
| S721STAT4 | | | | |

Please see the attached table for the name, host, application, and dilutions of antibodies used in immunoblot and immunohistochemical analyses.

GFP reporter assay. To assay the transcriptional activities of STAT3, cells at 75% confluence were transfected by 0.1 μg of pEZX-PF02 plasmid cloned with STAT3 response element of IL6 promoter upstream of eGFP reporter. The transfection was done by Lipofectamine 3000™ reagent as per the manufacturer's protocol (Thermofisher). After 24 hrs. of transfection, cells were treated with EGF and increasing doses of SIM for 24 hrs. GFP reporter activities were analyzed in PerkinElmer VictorX3 plate reader at Ex:Em=485/535 nm.

Acquisition, housing, and treatment of PDX mice. Grade 3 invasive ductal carcinoma tumor fragments were harvested from a 52-year-old female patient, genotyped for ER-, PR-, and Her2low, and were engrafted in the flank region of 6-to-7-week-old female NOD.Cg-Prkdcscid Il2rgtm1Wjl/SzJ mouse. These tumor-bearing mice were commercially purchased from Jackson Laboratories (Cat #TM00096). Since these mice are severely immunocompromised, special housing was required in a barrier room with restricted air flow and sterile foods. All animal studies were performed under an Institutional Animal Care and Use Committee-approved protocol (Study ID #). Both SIM-1 and SIM-2 were partly soluble to insoluble in aqueous solvent. Therefore, after careful literature study [25; 26], 5% DMSO was found to be an acceptable and safe solvent to dissolve SIM-1 and SIM-2 for the in-vivo study. For the treatment, 2 mg/Kg bwt doses of both SIM-1 and SIM-2 were dissolved in 5% DMSO and then administered intraperitoneally every alternate day for two weeks and followed up for additional two weeks. To ensure safety and toxicity, vital health parameters such as body weight, heart rate, and body temperature were recorded every alternate day for one month. Tumor growth was assessed every alternate day for one month by a digital caliper. Tumor volume was estimated by the formula 0.5× (length×width2). The treatment started once the tumor reached 4 mm in size with an approximate volume of ˜30 mm³. Mice received drugs for two weeks and left untreated for an additional two weeks to monitor the effect of drug withdrawal on the tumor size. On the penultimate day of the study, mice were injected with 100 μL of IRDye680-conjugated 2DG dye. The next day, mice were euthanized and then imaged in a Li—CoR infrared scanner to measure tumor size. After that, tumor tissues were harvested from the flank, washed in cold PBS, again imaged with a scalebar, and finally preserved in a −80° C. freezer for subsequent analyses such as genetic and immunoassays.

Statistical analysis. For two groups, an unpaired t-test was adopted with p<0.05. For more than one group, one-way ANOVA was adopted considering doses of SIMMPYRA as a single effector. Results are mean±SD of three independent experiments.

STAT3 is a primary target in breast cancer treatment. Activation of STAT3 has been implicated in the growth and progression of triple-negative breast tumors [27]. STAT3 was shown to induce TNBC tumor growth via upregulation of autophagy [28], inhibition of ROS and ROS-dependent apoptosis [29], and augmentation of immunosuppression [30]. Being a transcription factor, activated STAT3 enters into the nucleus and upregulates the transcription of many tumor-promoting genes such as c-myc, bcl-2, bcl-xl, cyclin D1, and survivin [31]. Moreover, STAT3 also upregulated expressions of cancer-promoting cytokines such as IL6 and RANTES in TNBC cells [32]. TNBC cells profusely express and secrete IL6 in the TNBC tumor microenvironment, which binds to IL6 receptors in an autocrine mode and supports tumor growth and progression. Previous reports suggest that STAT3-dependent production of IL6 [33] is required for the protection of tumor microenvironment and tumor growth during stress and starvation conditions. Moreover, STAT3-dependent upregulation of RANTES is also required for the sustenance of breast cancer cells under tamoxifen treatment condition suggesting the growth-supportive and drug-resistant role of RANTES in TNBC pathogenesis [34]. In fact, IL6 and RANTES synergize to produce the aggressive properties of TNBC cells [32]. Therefore, the activation of STAT3 has emerged as a critical factor in the regulation of immune response, induction of tumor-promoting genes, and the acquisition of drug resistance in TNBC cells. As a result, small molecular STAT3 inhibitors demonstrate great promise in the treatment of TNBC tumors. In this context, stattic and SD36 are the two most effective STAT3 inhibitors with a strong therapeutic potential in the treatment of TNBC. However, both stattic and SD36 downregulated STAT3 expression, suppressed Y705 phosphorylation of STAT3, and promoted the apoptosis of TNBC tumors at a range of 1 to 10 μM concentrations. The high CT50 of these compounds indicates that there could be a possible off-target effect and toxicity in immuno-compromised TNBC patients. That limits the enthusiasm for these compounds as therapeutic interventions in TNBC patients. Benzothiophene class of compounds can be potent STAT3 inhibitors. Structurally, stattic is a class of benzothiophene compounds having a dioxide substitution in sulfur and a nitrate substitution at C6 of the benzene ring. On the other hand, the present invention discloses SIM1 as a benzothiophene derivative with 4-(Methylamino)-4-oxobutanoic acid substitution at C3 of the thiophene ring and a chlorine substitution at C6 of the benzene ring. The present invention discloses a method starting with an in-silico analysis that revealed that with these two unique substitutions, SIM1 rendered a very strong binding to the SH2 domain of STAT3. In comparison to stattic (ΔG=−6.39 Cal/mole and full-fitness energy=−2897 Cal/mole), SIM1 showed much stronger binding (ΔG=−9.04 Cal/mole and full-fitness energy=−2984 Cal/mole) in the ligand binding pocket in SH2 domain of STAT3. On the other hand, the present invention also discloses SIM2, which has two substitutions at the thiophene ring, one acetamide group at C2 and other ethyl carboxylates at C3. In addition to that, two more substitutions were incorporated in the benzene ring, one chlorine, and an adjacent hydroxyimino group. The present invention further discloses that this compound also engaged with strong binding to STAT3 SH2 pocket and displayed a much stronger energy of interaction (ΔG=−8.15 Cal/mole and full-fitness energy=−2979 Cal/mole) compared to stattic. Interestingly, several experiments as shown in the Examples in the present disclosure hereinbelow, highlighted that SIMMPYRAs efficiently inhibited the activation of STAT3 at nanomolar concentration. First, a protein thermal shift assay revealed that both SIM1 and SIM2 significantly (>2° C.) shifted the melt curve of STAT3 as low as 5 nM concentrations. Second, a protein array analysis of active STATs indicated that 10 nM doses of both SIMMPYRAs selectively suppressed the level of Y705 phosphorylated STAT3, but not other phosphorylated STATs including STAT5 and STAT6, in TNBC tumor cells. Finally, a STAT3-responsive eGFP reporter assay revealed that both SIM1 and SIM2 inhibited STAT3 activation as low as 5 nM with IC50˜20 nM. Once STAT3 inhibitory roles of SIMMPYRAs were confirmed, next the present invention discloses an evaluation of the effects of these compounds in the apoptosis of TNBC tumor cells. Interestingly, an LDH release assay, an Annexin V/PI dual labeling analysis, and a TUNEL assay clearly demonstrated that increasing doses of both SIMMPYRAs strongly induced apoptosis in MDA-MB-468 TNBC cells as shown hereinbelow. The resultant CT50 is approximately 20 nM for both these compounds, which is much lower than CT50 values of known STAT3 inhibitors such as stattic and SD-36. Both stattic and SD-36 exhibited CT50 at the micromolar range. Moreover, similar analyses did not result in any apoptosis in HMEC control cells suggesting that the SIMMPYRAS specifically stimulated cytotoxicity only in TNBC cells, but not in healthy cells.

The present invention discloses that there are potential advantageous applications of SIMMPYRAs in other cancers and other STAT3-related diseases. STAT3 is profusely expressed in a wide range of human cancers [35]. Apart from TNBC, activation of STAT3 has been shown to be involved in the progression of a wide range of cancers including prostate cancer [36]; liver cancer [37]; brain cancers such as glioblastoma [38], and neuroblastoma [39]; leukemia [40]; lymphoma [41]; multiple myeloma [42]; colorectal cancer [15]; and lung cancer [43]. Therefore, the therapeutic potentials of SIMMPYRAs are not limited to the treatment of TNBC tumors, but other forms of cancers. Further, apart from cancer, the present invention proposes that the activation of STAT3 by mammalian target of rapamycin (mTOR) kinase and subsequent productions of IL6 and RANTES in muscle tissue augment muscle fatigue after treadmill exercise. In fact, consistent with previous studies [44; 45], the present invention provides that ME/CFS patients displayed upregulated levels of IL6 and RANTES in their plasma suggesting the role of STAT3 activation in ME/CFS pathogenesis. Therefore, the present invention proposes the use of SIMMPYRAs in the amelioration of post-exertional muscle fatigue. Taken together, the present invention as disclosed herein identifies two selective inhibitors of STAT3 that efficiently inhibit STAT3 activation at exceptionally low doses, strongly downregulated expressions of STAT3-dependent tumorigenic genes, and promote cytotoxicity in TNBC cells.

The specific formulation, design, and functionality of the components will depend on the intended method of administration and delivery of SIM1 and/or SIM2, and the desired release kinetics of the inhibiting small-molecule inhibitor targeting STAT3.

The invention will be further explained by the following Examples, which are intended to be purely exemplary of the invention and should not be considered as limiting the invention in any way.

### EXAMPLES

### Example 1: Identification and Synthesis of SIMMPYRA1 (SIM1) and SIMMPYRA2 (SIM2)

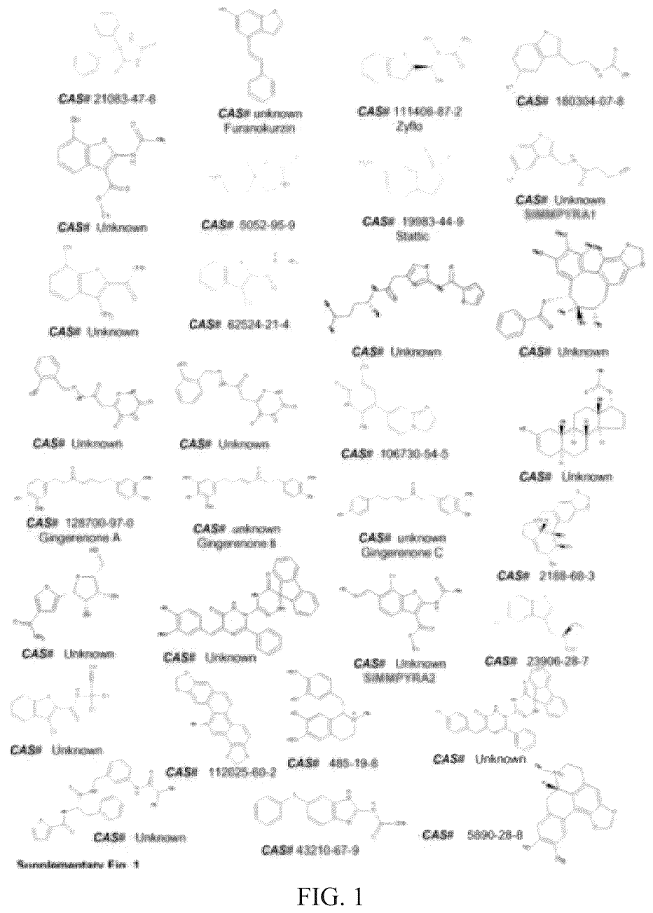

This example exemplifies an embodiment of a method of preparation consisting of the steps of identification and synthesis of the novel small-molecule inhibitor targeting STAT3 activation as disclosed in the present invention by employing an in-silico-based high-throughput screening analysis where two known STAT3 inhibitors named SD36 (PDB ID: 6NJS) and stattic were included as positive controls. Both these compounds promote the death of TNBC cells at IC50>5 μM, i.e., in the micromolar range. Therefore, the present invention identifies new inhibitors that could induce apoptosis of TNBC cells at nanomolar (nM) concentration. A compound with a benzothiophene backbone [18] can be a potent STAT3 inhibitor. Therefore, compounds were selected with benzothiophene backbone, and twenty-five unknown structural analogs of STAT3 inhibitors were tested for docking with STAT3 in the Swiss-Dock server to identify potential compounds that are novel small-molecule inhibitors targeting STAT3 activation via this in-silico approach to dock 32 structurally related compounds which are listed in Table 2, and their structural info such as unique database number and mol2 structural file were derived from the ZINC docking database (Refer to FIG. 1).

TABLE 2
List of compounds selected for in-silico screening. A list of 32
compounds, their unique ID generated by the ZINC database
(https://zinc.docking.org), molecular formula, structural schema, free energy
change of docking, and total fitness energy of docking were displayed.

| | | | Molecular | | E full |
|---|---|---|---|---|---|
| Rank | ZINC ID | Structure | Formula | ΔG | fitness |
| L1 | ZINC16916510 | 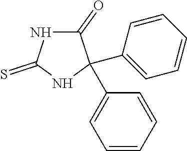 [SMILES: O=C1NC(=S)NC1(c1ccccc1)c1ccccc1] | C15H12N2OS | N.A. | N.A. |
| L2 | ZINC95553268 | 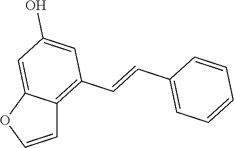 [SMILES: Oc1cc(/C=C/c2ccccc2)c2ccoc2c1] | C16H12O2 | −6.6318097 | −2918.4573 |
| L3 | ZINC850 | 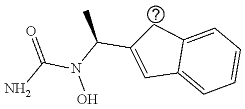 [SMILES: C[C@@H](C1=Cc2ccccc2C1)N(O)C(N)=O] | C11H12N2O2S | −6.9032493 | −2938.1794 |
| L4 | ZINC6676 | 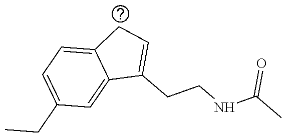 [SMILES: CCc1ccc2c(c1)C(CCNC(C)=O)=CC2] | C14H17NOS | −6.9023595 | −2934.8162 |
| L5 | ZINC33484 | 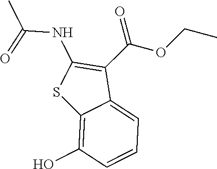 [SMILES: CCOC(=O)c1c(NC(C)=O)sc2c(O)cccc12] | C13H13NO4S | −6.6145134 | −2939.4224 |
| L6 | ZINC14087 | 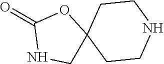 [SMILES: O=C1NCC2(CCNCC2)O1] | C7H12N2O2 | −6.3761163 | −3002.6362 |
| L7 | ZINC162014 | 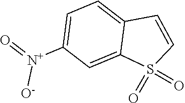 [SMILES: O=[N+]([O-])c1ccc2c(c1)S(=O)(=O)C=C2] | C8H5NO4S | −6.3957686 | −2896.678 |
| L8 | ZINC147736 | 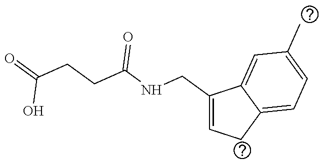 [SMILES: Cc1ccc2c(c1)C(CNC(=O)CCC(=O)O)=CC2] | C13H12ClNO3S | −9.044236 | −2984.8464 |
| L9 | ZINC142201 | 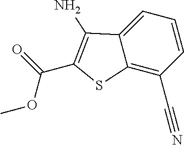 [SMILES: COC(=O)c1sc2c(C#N)cccc2c1N] | C11H8N2O2S | −6.7031393 | −2923.0786 |
| L10 | ZINC35754 | 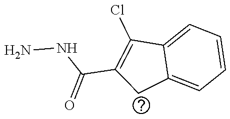 [SMILES: NNC(=O)C1=C(Cl)c2ccccc2C1] | C9H7ClN2OS | −6.327298 | −2925.1091 |
| L11 | ZINC253488033 | 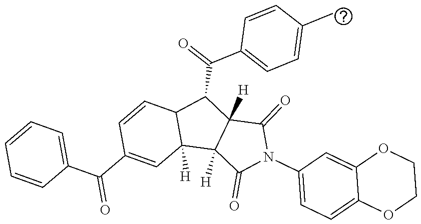 [SMILES: Cc1ccc(C(=O)[C@H]2C3C=CC(C(=O)c4ccccc4)=C[C@@H]3[C@@H]3C(=O)N(c4ccc5c(c4)OCCO5)C(=O)[C@H]32)cc1] | C32H23ClN2O6 | −8.740316 | −2827.1772 |
| L12 | ZINC17654726 | 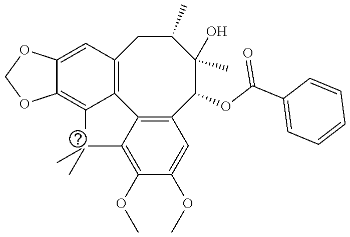 [SMILES: COc1cc2c3c(c1OC)C(C)(C)c1c4c(cc(c1-3)C[C@H](C)[C@@](C)(O)[C@@H]2OC(=O)c1ccccc1)OCO4] | C30H32O9 | −6.9075537 | −2762.731 |
| L13 | ZINC19792227 | 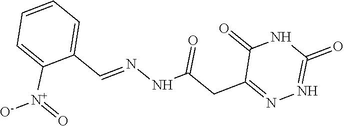 | C12H10N6O5 | −7.3778996 | −2912.9912 |
| L14 | ZINC33342950 | 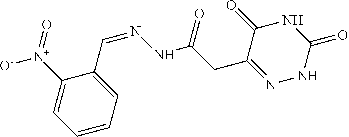 [SMILES: O=C(Cc1n[nH]c(=O)[nH]c1=O)N/N=C\c1ccccc1[N+](=O)[O-]] | C12H10N6O5 | −7.2466617 | −2914.281 |
| L15 | ZINC1851 | 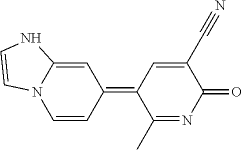 [SMILES: CC1=NC(=O)C(C#N)=C/C1=C1/C=CN2C=CNC2=C1] | C14H10N4O | −7.7135544 | −2924.7854 |
| L16 | ZINC1843 | 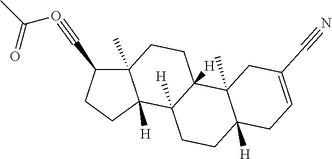 | C22H31NO2 | −6.5413327 | −2892.8655 |
| L17 | ZINC1531844 | 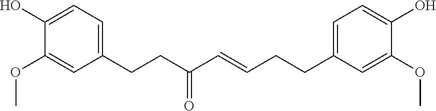 [SMILES: COc1cc(CC/C=C/C(=O)CCc2ccc(O)c(OC)c2)ccc1O] | C21H24O5 | −7.2997074 | −2897.4375 |
| L18 | ZINC13377938 | 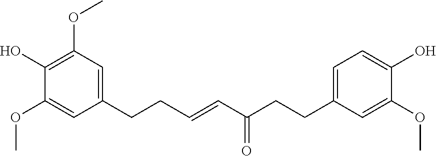 [SMILES: COc1cc(CCC(=O)/C=C/CCc2cc(OC)c(O)c(OC)c2)ccc1O] | C22H26O6 | −8.046166 | −2890.4883 |
| L19 | ZINC13341090 | 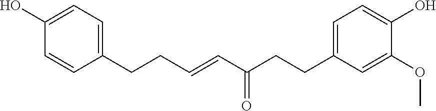 [SMILES: COc1cc(CCC(=O)/C=C/CCc2ccc(O)cc2)ccc1O] | C20H22O4 | −7.71339 | −2914.483 |
| L20 | ZINC24 | 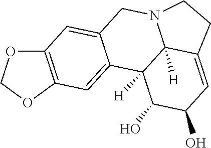 | C16H17NO4 | −7.6604733 | −2909.564 |
| L21 | ZINC28261447 | 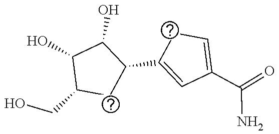 [SMILES: NC(=O)C1=CCC([C@@H]2C[C@H](CO)[C@H](O)[C@@H]2O)=C1] | C10H13NO4S2 | −6.8476186 | −2903.0212 |
| L22 | ZINC1641980 | 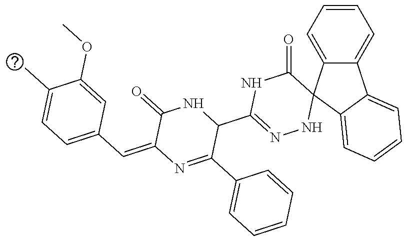 [SMILES: COc1cc(/C=C2/N=C(c3ccccc3)C(C3=NNC4(C(=O)N3)c3ccccc3-c3ccccc34)NC2=O)ccc1C] | C32H24N6O4 | −8.917717 | −2791.3833 |
| L23 | ZINC53354 | 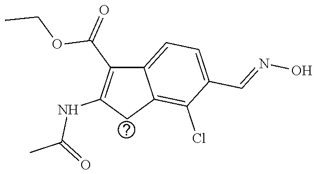 [SMILES: CCOC(=O)C1=C(NC(C)=O)Cc2c1ccc(/C=N/O)c2Cl] | C14H13ClN2O4S | −8.15172 | −2979.9116 |
| L24 | ZINC161531 | 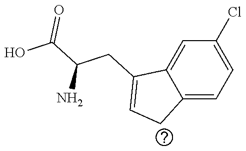 [SMILES: N[C@H](CC1=CCc2ccc(Cl)cc21)C(=O)O] | C11H10ClNO2S | −7.1535587 | −2917.7834 |
| L25 | ZINC66854 | 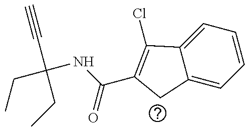 [SMILES: C#CC(CC)(CC)NC(=O)C1=C(Cl)c2ccccc2C1] | C16H16ClNOS | −6.756643 | −2943.623 |
| L26 | ZINC706 | 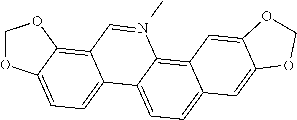 [SMILES: C[n+]1cc2c3c(ccc2c2ccc4cc5c(cc4c21)OCO5)OCO3] | C20H14NO4+ | −7.228487 | −2864.7776 |
| L27 | ZINC895630 | 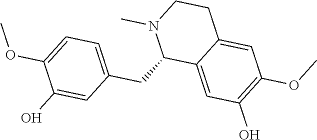 | C19H23NO4 | −6.848183 | −2897.2205 |
| L28 | ZINC1641976 | 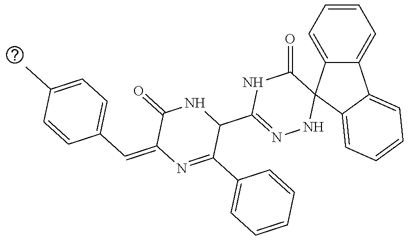 [SMILES: Cc1ccc(/C=C2/N=C(c3ccccc3)C(C3=NNC4(C(=O)N3)c3ccccc3-c3ccccc34)NC2=O)cc1] | C31H21ClN6O2 | −8.589391 | −2786.7566 |
| L29 | ZINC257440150 | 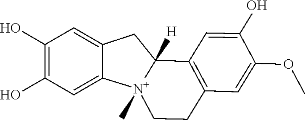 [SMILES: COc1cc2c(cc1O)[C@H]1Cc3cc(O)c(O)cc3[N@@+]1(C)CC2] | C18H20NO4+ | −7.4611473 | −2917.4097 |
| L30 | ZINC898481 | 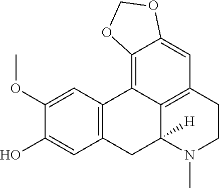 | C19H19NO4 | −6.584012 | −2875.4192 |
| L31 | ZINC402911 | 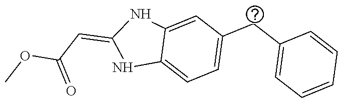 [SMILES: COC(=O)/C=C1\Nc2ccc(Cc3ccccc3)cc2N1] | C15H13N3O2S | −6.753424 | −2980.0608 |

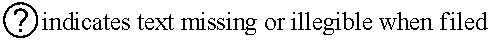

Some of the afore-listed compounds have only SMILES (simplified molecular-input line-entry system) linear input as structural information and were converted to mol2 format with three-dimensional atomic coordinates with the help of the Open Babel converting tool (cheminfo.org) before setting up in the docking server. Using a rigid body protein-ligand docking strategy in the SwissDock online tool, the present invention predicted the interaction of all 32 compounds in the SH2 inhibitory domain of STAT3 at the molecular level. While conducting docking analysis, the present invention in this example also docked two known STAT3 inhibitors named stattic and SD36 in the SH2 domain of STAT3 as positive controls. Structural analogs furanokurzin, a potent cholinesterase inhibitor [46], and zyflo, a selective leukotriene inhibitor [47], and S-22153 (melatonin antagonist) were included as negative controls.

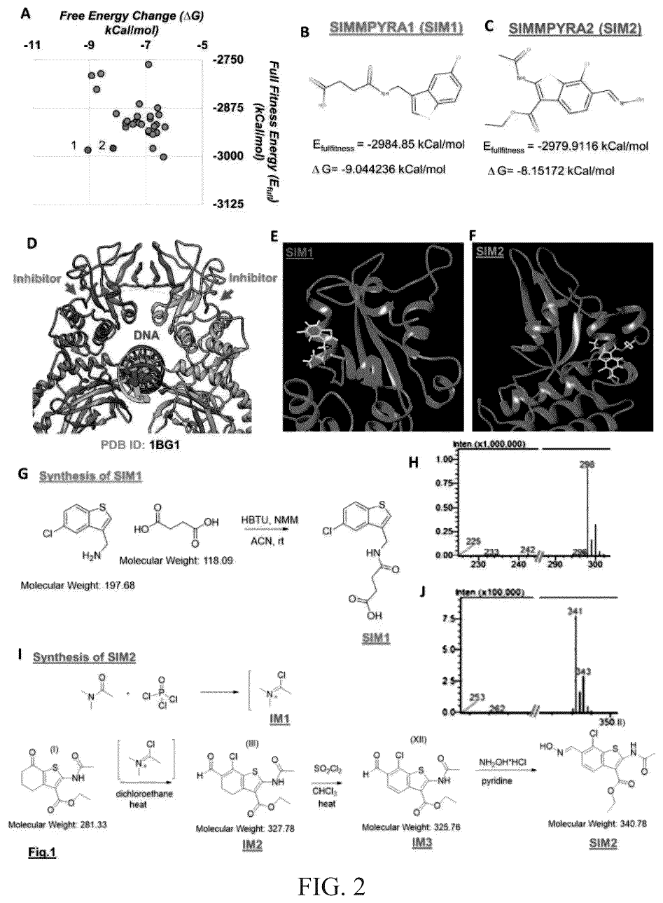

In reference to human STAT3 (Protein Data Bank ID code 1BG1) as a template, the auto-dock module of SwissDock server generated 100 probable structures for each compound. Combining total free energy (ΔG) and total fitness energy (Eₜₒₜ) derived from the most suitable docking pose of each compound, a scatter plot analysis was conducted to rank all 32 compounds (Refer to FIG. 2A). Based on that analysis, two novel compounds named 4-(((5-chlorobenzo[b]thiophen-3-yl)methyl)amino)-4-oxobutanoic acid (Refer to FIG. 2B) referred to as SIMMPYRA-1 (SIM1) and Ethyl (E)-2-acetamido-7-chloro-6-((hydroxyimino)methyl)benzo[b]thiophene-3-carboxylate (Refer to FIG. 2C) referred to as SIMMPYRA-2 (SIM2) were identified that can effectively bind to the SH2 inhibitory domain of STAT3 (Refer to FIG. 2D). Subsequently, the most stable docking poses of both these compounds were displayed at 5 Å resolution (Refer to FIG. 2E and FIG. 2F) that showed a strong binding in the SH2 domain of STAT3.

Next, the method of the present invention as exemplified in this example synthesized these two compounds as described below.

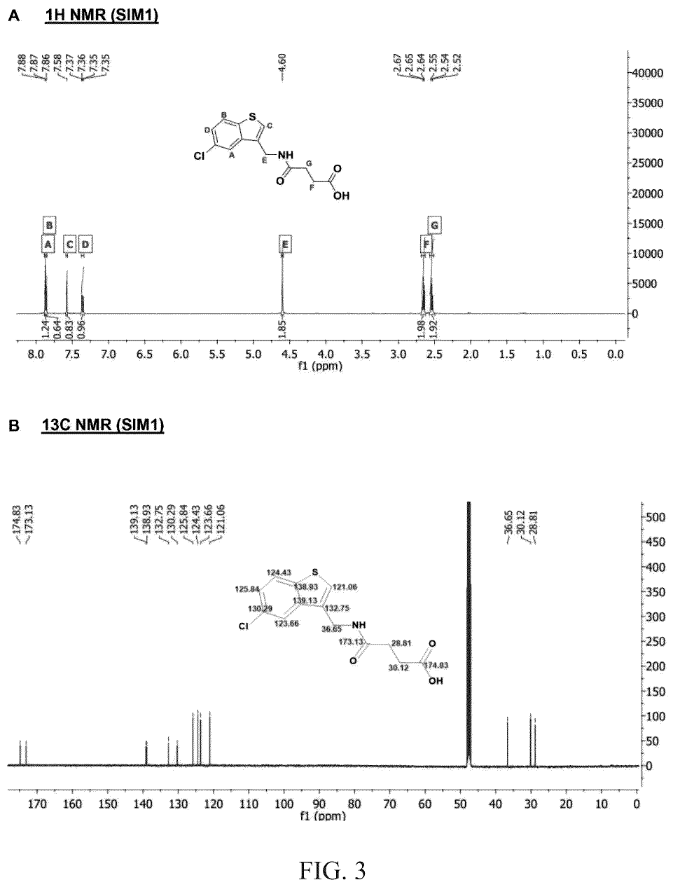

Synthesis of SIM1: To synthesize SIM1, the present invention first adopted the HBTU (Hexafluorophosphate Benzotriazole Tetramethyl Uronium) coupling process in which succinic acid along with HBTU and N-methylmorpholine (NMMV4) was dissolved in dry acetonitrile (ACN) under nitrogen followed by the addition of (5-Chloro-1-benzothien-3-yl) methanamine with constant stirring for hrs. at room temperature (Refer to FIG. 2G). Next, the resulting residue was dissolved with chloroform, washed with 1M ammonium chloride and brine solution, dried with magnesium sulfate, concentrated, and purified using column chromatography as described hereinabove. The subsequent characterization by the LC-MS study identified a base peak at e/z 298 (Refer to FIG. 2H). To confirm the structural conformation of the synthesized molecule ¹H-NMR (FIG. 3A) and 1³C-NMR (FIG. 3B) were performed.

Synthesis of SIM2: SIM2 synthesis involves a multi-step process and is summarized with a schema (Refer to FIG. 2I). First, the Vilsmeier reagent was prepared as described hereinabove (shown as intermediate1 or IM1). The second step was to synthesize ethyl 2-acetamido-7-chloro-6-formyl-4,5-dihydrobenzo[b]thiophene-3-carboxylate, which was shown as intermediate 2 or IM2. Briefly, a solution of ethyl 2-acetamido-7-oxo-4,5,6,7-tetrahydrobenzo[b]thiophene-3-carboxylate dissolved in dry dichloroethane was added to Vilsmeier reagent, stirred at room temperature, refluxed at 75° C., washed, and then the resultant organic layer was dried with magnesium sulfate and concentrated. The third step was the synthesis of ethyl 2-acetamido-7-chloro-6-formylbenzo[b]thiophene-3-carboxylate or IM3. IM2 along with sulfuryl chloride (SO₂Cl₂) in dichloromethane (DCM) solution was stirred in dry chloroform (CHCl₃) at RT for 30 minutes, refluxed at 65° C. for 1.5 hours, cooled, washed with DI water, the resultant organic layer was dried with magnesium sulfate and then concentrated to yield IM3 as an off-white powder.

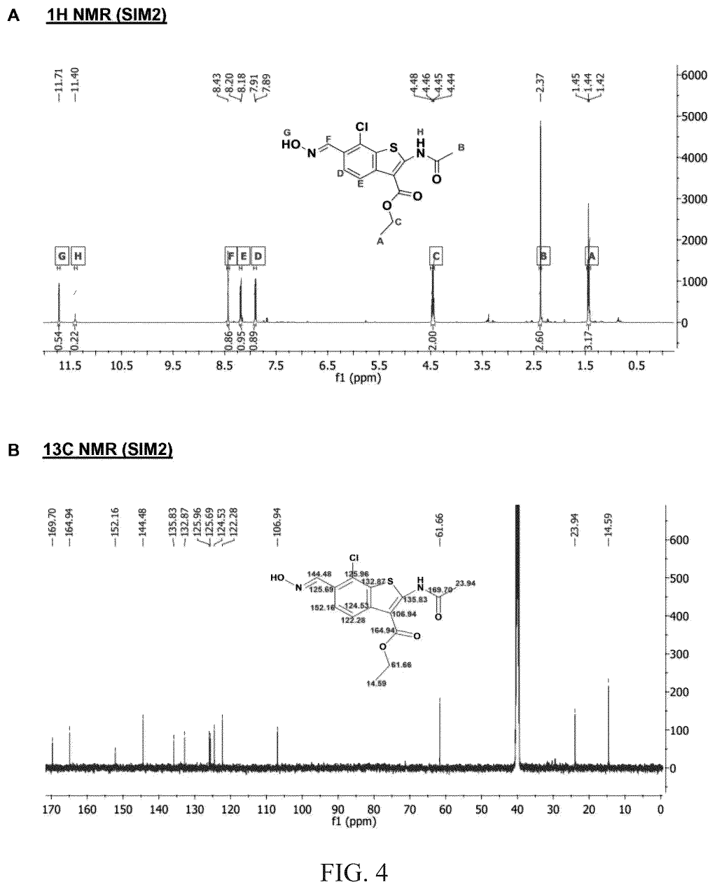

The final step was the synthesis of ethyl (E)-2-acetamido-7-chloro-6-((hydroxyimino)methyl)benzo[b]thiophene-3-carboxylate or SIMMPYRA-2. Briefly, IM3 was dissolved in pyridine, and combined with NH2OH·HCl, stirred at RT for 24 h, poured dropwise into DI water to form a precipitate, filtered, and dried as a white powder. Subsequently, LC-MS characterization of SIMMPYRA-2 resulted in an e/z base peak at 341 (Refer to FIG. 2J). Subsequent ¹HNMR (FIG. 4A) and ¹³C NMR (FIG. 4B) were performed to confirm the structural conformation of SIM-2. Taken together, the medium-throughput screening analyses as disclosed in the present invention identified SIMMPYRA-1 (SIM1 or SIM-1 used interchangeably throughout) and SIMMPYRA-2 (SIM2 or SIM-2 used interchangeably throughout) as potential inhibitors of STAT3, and the present invention also synthesized these two compounds for the subsequent cellular assay to validate their STAT3 inhibitor role.

### Example 2: Evaluation of SIMMPYRAs as STAT3 Inhibitors

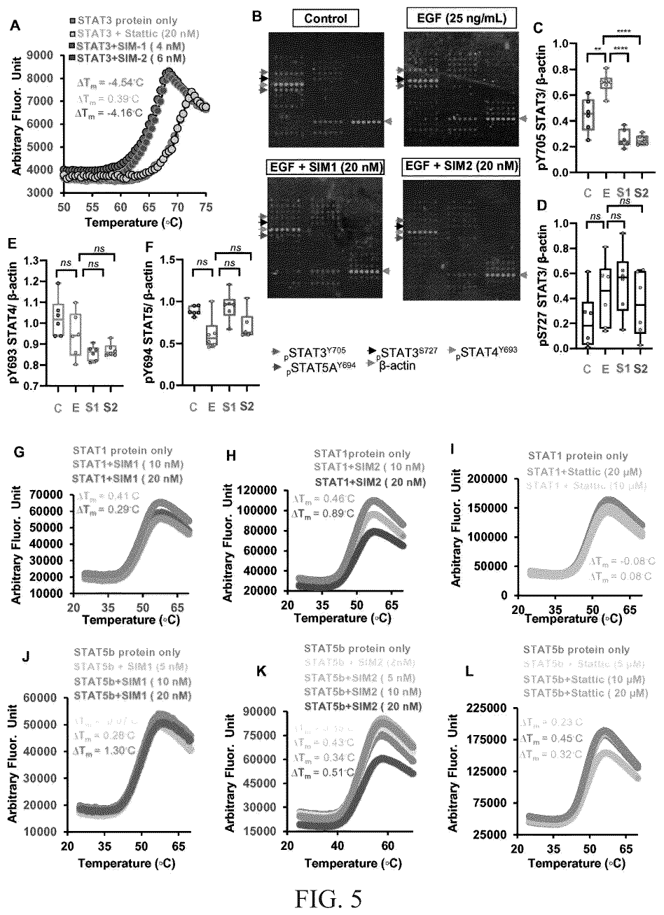

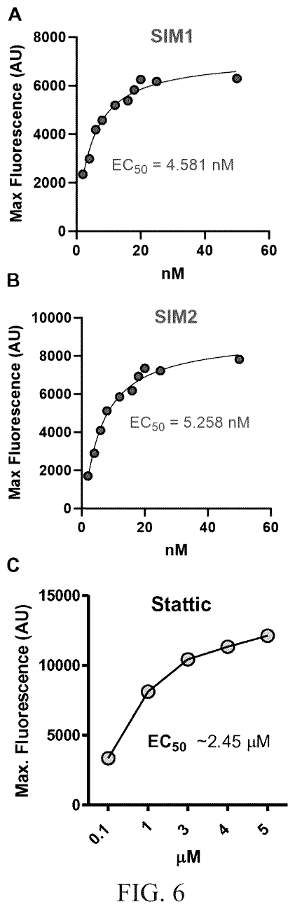

This example provides a representative study of the present disclosure to test if SIM1 and SIM2 served as ligands of STAT3. Therefore, the present invention adopted a protein thermal shift assay in which 1p g of recombinant human STAT3 protein (active motif) was incubated with increasing doses of SIM1 and SIM2 followed by the execution of melting analysis starting from 25 to 95° C. as described elsewhere [48]. Interestingly, the present invention observed that both SIM-1 and SIM-2 caused significant (cut-off ΔTₘ=Tₘ^{protein}−Tₘ^{protein+ligand}=2° C.) shifts of STAT3 protein as low as 4 nM (ΔTₘ=−4.54° C.) and 6 nM (ΔTₘ=−4.16° C.) concentrations (Refer to FIG. 5A) suggesting that both these compounds serve as strong ligands of STAT3. Stattic, a known STAT3 inhibitor and a positive control having a similar benzothiophene backbone, was observed to produce a negligible shift (ΔTₘ=0.39° C.) even at 20 nM concentration. Moreover, a non-linear hyperbolic binding curve of maximum fluorescence versus increasing doses further revealed that both SIM1 and SIM2 have very high affinity to STAT3 with EC₅₀=4.581 (FIG. 6A) and 5.258 nM (FIG. 6B) respectively, whereas the positive control stattic displayed much lower affinity (EC₅₀˜2000 nM) (FIG. 6C).

Next, the present invention evaluated if these compounds as disclosed in the present invention as small-molecule inhibitors targeting STAT3 activation, inhibited the activation of STAT3, but not other STATs. To validate, a protein microarray analysis was performed. In this analysis, MDA-MB-468 TNBC cancer cells were treated with vehicle (DMSO only), epidermal growth factor or EGF (25 ng), EGF and SIM1 or EGF+SIM1 (5 nM) and EGF and SIM2 or EGF+SIM2 (5 nM) for 6 hrs. Although TNBC cells have depleted HER2 receptors, EGF could still stimulate these cells via the EGF receptor (EGFR) [49]. After that, cell lysates were employed on commercially available protein array membranes (Full moon Biosystems) embedded with phospho-serine (pS) and phospho-tyrosine (pY) antibodies of STAT1, STAT2, STAT3, STAT4, STAT5, and STAT6. After the incubation, membranes were imaged in Li-cor Odyssey Sa imager and relative densities of all spots were quantified after normalizing with housekeeping proteins by ImageJ software as described under method section (Refer to FIG. 5B). Interestingly, both SIM1 and SIM2 strongly and selectively suppressed the levels of pY705 STAT3 (Refer to FIG. 5C), but not pS727 STAT3 (Refer to FIG. 5D), pY693STAT4 (Refer to FIG. 5E), pY694STAT5 (Refer to FIG. 5F) suggesting that both SIM1 and SIM2 selectively inhibit the activation and phosphorylation at Y705 residue of STAT3, but not the other STATs, at nM concentration.

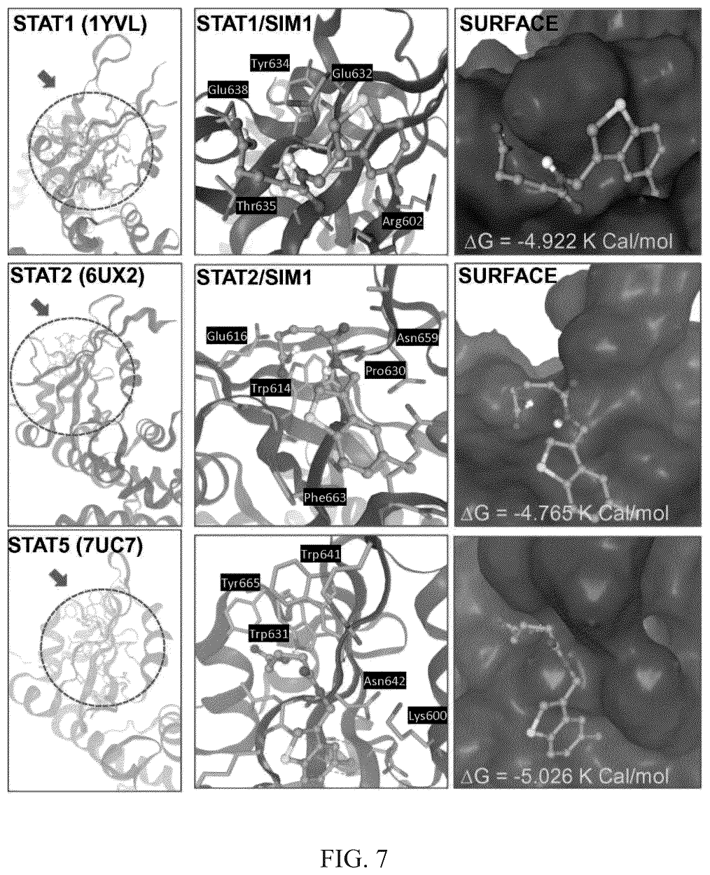

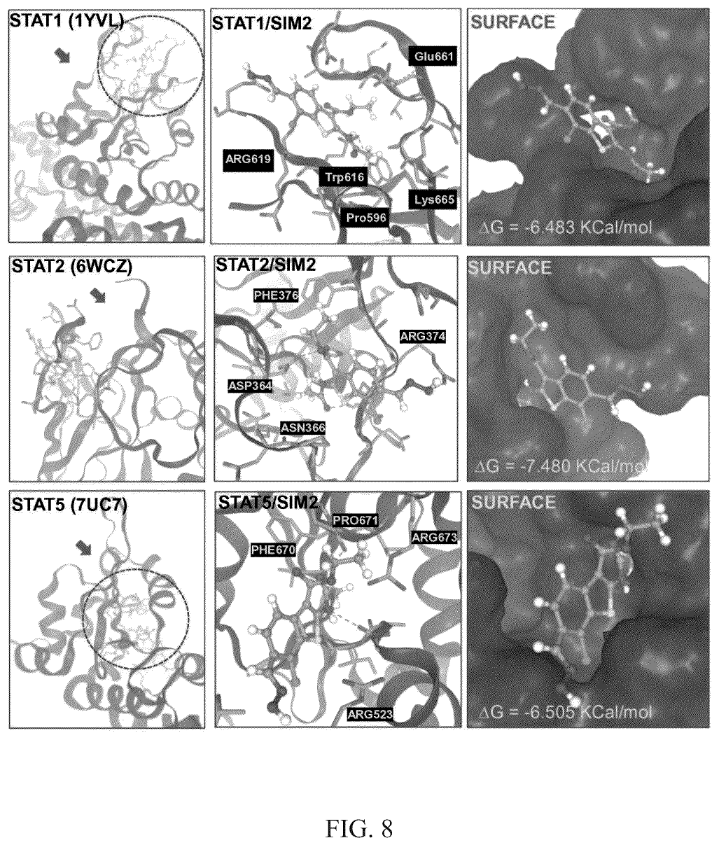

Although the disclosed in-silico rigid body docking analyses of the present example demonstrated that both SIM-1 (FIG. 7) and SIM-2 (FIG. 8) can be docked in the ligand binding SH2 domains of STAT1 (PDB ID: 1YVL), STAT2 (PDB ID: 6UX2/6WCZ), and STAT5 (PDB ID: 7UC7) with variable efficiencies, subsequent biochemical validation is warranted.

To confirm the result, PTS assay of STAT1 with SIM-1 (FIG. 5G), SIM-2 (FIG. 5H), and stattic (FIG. 5I) was performed. Interestingly, nM (ΔTₘ=Tₘ^{protein+iigand}−Tₘ^{protein only}=0.41° C.) and 20 nM of SIM-1 (ΔTₘ=0.29° C.) were unable to produce any significant shift in the melting curve of STAT1 (FIG. 5G). Similarly, 10 nM (ΔTₘ=0.46° C.) and 20 (ΔTₘ=0.89° C.) nM of SIM-2 also failed to shift the melting curve of STAT1(FIG. 5H). Positive control of STAT3 binding small molecule stattic (10 and 20 μM) also did not produce any shift in the melting curve of STAT1(FIG. 5I). Collectively, these results suggest that SIM-1 and SIM-2 do not exhibit any interaction with STAT1 at nM concentration range. Similar results were observed while performing the binding assay with STAT5b. Increasing doses of SIM-1 (FIG. 5J), SIM-2 (FIG. 5K), and stattic (FIG. 5L) were unable to produce any significant shift in the melting curve of STAT5b. Taken together, the results of the present example demonstrated that both SIM-1 and SIM-2 displayed strong binding to STAT3 at nM concentration range.

### Example 3: SIMMPYRAs Downregulated STAT3-Dependent Tumor-Promoting Factors Such as IL6 and RANTES

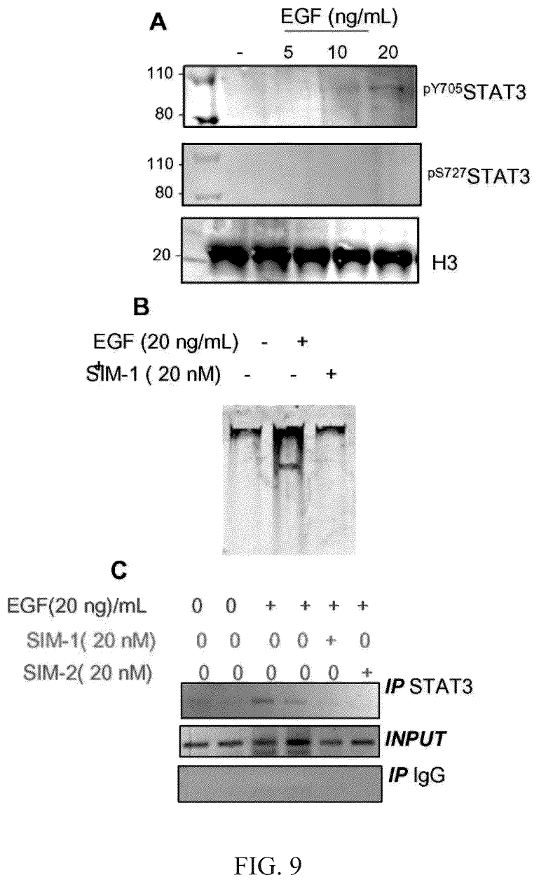

This example provides a representative experiment of the present invention first explored if SIM-1 and SIM-2 inhibit STAT3 activation in a cell-bound system. Activation of STAT3 directly regulates expressions of a wide range of inflammatory cytokines, tumor-promoting factors, and anti-apoptotic proteins in TNBC cells. Upregulation of these factors often promotes tumor growth, survival, and progression of TNBC. This example Immunoblot analyses revealed that upon stimulation with 5, 10, and 20 ng/mL EGF for 5 hrs, MDA-MB-468 cells displayed strong upregulation of Y705 phosphorylation, but not S727 phosphorylation of STAT3 (FIG. 9A) in the nuclear fraction.

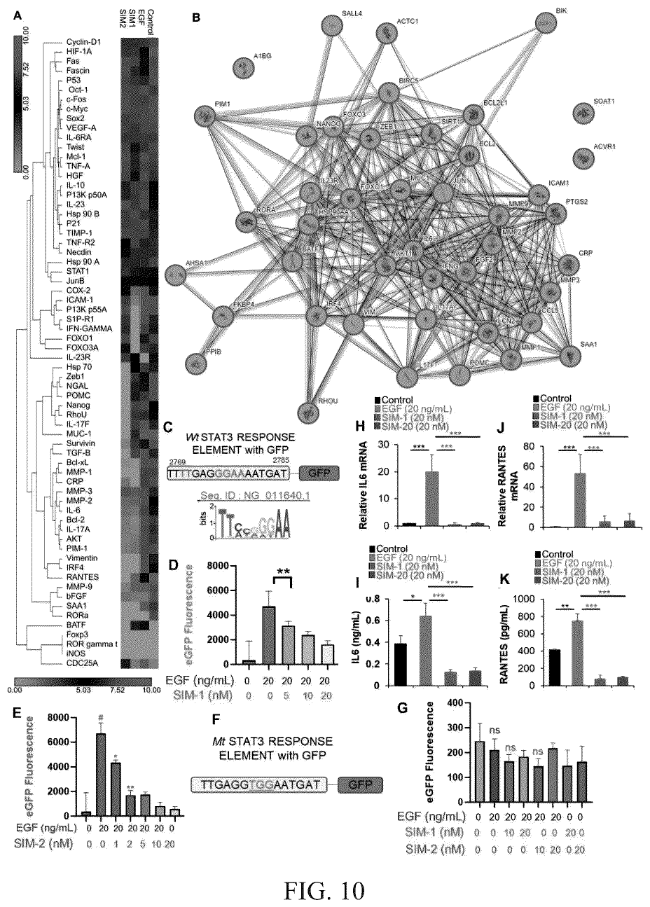

To understand the molecular regulation of SIMMPYRAS in modulating STAT3 activity, the following studies in MDA-MB-468 cells were adopted in this example. First, an electrophoretic mobility shift assay (or EMSA) in the nuclear extract of SIM-1-treated EGF-stimulated MDA-MB-468 cells followed by probing with consensus STAT3 oligonucleotide or SEQ ID NO: 1 revealed that SIM-1 markedly inhibited the DNA binding activity of STAT3 (FIG. 9B) suggesting that SIM-1 truly inhibited the nuclear translocation and subsequent activation of STAT-3. Second, SIM-1 and SIM-2 treatments significantly attenuated the expressions of STAT-3-dependent tumor-promoting genes. A cDNA-based gene array analysis (FIG. 10A) was performed in which 66 previously described [50] STAT3-dependent tumor-promoting genes (Refer to Table 3) were assayed to explore if SIM1 and SIM2 indeed inhibited expressions of these genes in EGF-stimulated TNBC cells.

TABLE 3
The list of 66 STAT3-dependent genes and primers.

| S. | | | | Product |
|---|---|---|---|---|
| No. | Genes | Accession and base pair | Forward and Reverse primers | length |
| 1. | c-Fos | 208 bp linear mRNA | GGTACTCTGTGGGTTGCTCC or SEQ | 210 |
| | | Accession: AH003773.2 | ID NO: 6 | |
| | | GI: 1049010621 | GTGAGCTGCCAGGATGAACT or SEQ | |
| | | | ID NO: 7 | |
| 2. | HIF-1a | 3,819 bp linear mRNA | TCAAAGTCGGACAGCCTCAC or SEQ | 252 |
| | | Accession: NM 181054.3 | ID NO: 8 | |
| | | GI: 1678209109 | ATCCATTGATTGCCCCAGCA or SEQ | |
| | | | ID NO: 9 | |
| 3. | c-Myc | 2,564 bp linear mRNA | GCCTCTTCACGGGCTAGAAA or SEQ | 362 |
| | | Accession: GU170215.1 | ID NO: 10 | |
| | | GI: 301030820 | TAAAGCGGGACTGAACACCC | |
| | | | or | |
| | | | SEQ ID NO: 11 | |
| 4 | Sox2 | 954 bp linear mRNA | AACCAGCGCATGGACAGTTA or | 278 |
| | | Accession: OP680447.1 | SEQ ID NO: 12 | |
| | | GI: 2430294553 | GACTTGACCACCGAACCCAT or SEQ | |
| | | | ID NO: 13 | |
| 5. | Nanog | 2,114 bp linear mRNA | GGAGCCTAATCAGCGAGGTT or SEQ | 421 |
| | | Accession: AB093576.1 | ID NO: 14 | |
| | | GI: 31338865 | GCCAGAGACGGCTTCTATCA or SEQ | |
| | | | ID NO: 15 | |
| 6. | Twist | 2,870 bp linear DNA | CCCAGTCCACCTCGATTTCC or SEQ | 222 |
| | | Accession: X91662.1 | ID NO: 16 | |
| | | GI: 999455 | AAACGGTCCTTACCCGTGAC or SEQ | |
| | | | ID NO: 17 | |
| 7 | Zeb1 | 6,195 bp linear mRNA | GGGGGAAAAGCGATCCTGAA or | 431 |
| | | Accession: | SEQ ID NO: 18 | |
| | | NM 001323671.2 | CTGTGTCATCCTCCCAGCAG or SEQ | |
| | | GI:1677538191 | ID NO: 19 | |
| 8. | P53 | 2,451 bp linear mRNA | AGCACTGTCCAACAACACCA or SEQ | 193 |
| | | Accession: AB082923.1 | ID NO: 20 | |
| | | GI: 23491728 | CTTCAGGTGGCTGGAGTGAG or SEQ | |
| | | | ID NO: 21 | |
| 9. | Oct-1 | 7,208 bp linear mRNA | CTTAAGGCGCGAAATCTGCC or SEQ | 509 |
| | | Accession: | ID NO: 22 | |
| | | XM_054337095.1 | GCGATCTCGGAGAAGTCCAG | |
| | | | or | |
| | | GI: 2462510205 | SEQ ID NO: 23 | |
| 10. | Bcl-2 | 720 bp linear mRNA | GAACTGGGGGAGGATTGTGG or | 291 |
| | | Accession: EU287875.1 | SEQ ID NO: 24 | |
| | | GI: 161277340 | TTGTGGCTCAGATAGGCACC or SEQ | |
| | | | ID NO: 25 | |
| 11. | Mcl-1 | 8,253 bp linear DNA | GTGTCAAGACTCTCAGACGAGAT or | 245 |
| | | Accession: AF198614.1 | SEQ ID NO: 26 | |
| | | GI: 7582270 | AGAGGGCATAGTTGGGGACT | |
| | | | or | |
| | | | SEQ ID NO: 27 | |
| 12. | Bcl-xL | 926 bp linear mRNA | TGTCTCAGAGCAACCGGGA or SEQ | 228 |
| | | Accession: Z23115.1 | ID NO: 28 | |
| | | GI: 510900 | CATCCAAACTGCTGCTGTGCG or | |
| | | | SEQ ID NO: 29 | |
| 13. | Survivin | 14,796 bp linear DNA | CCTGCTTTGTCCCCATCGAG or SEQ | 221 |
| | | Accession: U75285.1 | ID NO: 30 | |
| | | GI: 2315862 | GAGGCTGGCCAGAGAAGAC or SEQ | |
| | | | ID NO: 31 | |
| 14. | Fas | 7,515 bp linear mRNA | CGGAGGCATCAACCCAGATT or SEQ | 463 |
| | | Accession: U26644.1 | ID NO: 32 | |
| | | GI: 1049052 | GATGGTGGTGTAGACCTTCCG | |
| | | | or | |
| | | | SEQ ID NO: 33 | |
| 15. | Hsp 70 | 2,691 bp linear DNA | GACCAACACCCTTCCCACC or SEQ | 209 |
| | | Accession: M11717.1 | ID NO: 34 | |
| | | GI: 184416 | TTCTGAGCCAATCACCGAGC or SEQ | |
| | | | ID NO: 35 | |
| 16. | Hsp 90 α | 2,752 bp linear | GGCAGAGACCATCCAAGAAGT or | 216 |
| | | transcribed-RNA | SEQ ID NO: 36 | |
| | | Accession: NR 073383.1 | ACACAGCGCACATAGAGCTT or SEQ | |
| | | GI: 409971447 | ID NO: 37 | |
| 17. | Hsp 90 β | 2,017 bp linear mRNA | GGCCAACTCAGCTTTTGTGG or SEQ | 370 |
| | | Accession: AF275719.1 | ID NO: 38 | |
| | | GI: 9082288 | CATGGTGGAGTTGTCCCGAA or SEQ | |
| | | | ID NO: 39 | |
| 18. | Cyclin-D1 | 4,245 bp linear mRNA | CTGATTGGACAGGCATGGGT or SEQ | 322 |
| | | Accession: BC023620.2 | ID NO: 40 | |
| | | GI: 33991562 | GTGCCTGGAAGTCAACGGTA or SEQ | |
| | | | ID NO: 41 | |
| 19. | IL-10 | 691 bp linear mRNA | TGCAAAAGAAGGCATGCACAG or | 365 |
| | | Accession: BC104253.1 | SEQ ID NO: 42 | |
| | | GI: 74355811 | TCTTCAGGTTCTCCCCCAGG or SEQ | |
| | | | ID NO: 43 | |
| 20. | IL-23 | 1,037 bp linear mRNA | CCCAAGGACTCAGGGACAAC or | 274 |
| | | Accession: NM 016584.3 | SEQ ID NO: 44 | |
| | | GI: 1519246469 | TGGAGGCTGCGAAGGATTTT or SEQ | |
| | | | ID NO: 45 | |
| 21. | TGF-β | 1,448 bp linear mRNA | CGGATCTCTTCCTGCTCGAC or SEQ | 462 |
| | | Accession: M60316.1 | ID NO: 46 | |
| | | GI: 339563 | CACAGTAGTAGGCGGCGTAG or | |
| | | | SEQ ID NO: 47 | |
| 22 | COX-2 | 3,387 bp linear mRNA | ATCTACGGTTTGCTGTGGGG or SEQ | 341 |
| | | Accession: M90100.1 | ID NO: 48 | |
| | | GI: 181253 | AAAGGTGTCAGGCAGAAGGG | |
| | | | or | |
| | | | SEQ ID NO: 49 | |
| 23 | MMP-1 | 1,817 bp linear mRNA | CTAGGGCACTGGCGAAACC or SEQ | 352 |
| | | Accession: AF219624.1 | ID NO: 50 | |
| | | GI: 12698337 | ACTGAAACGCTCTGATGGCA or SEQ | |
| | | | ID NO: 51 | |
| 24 | MMP-2 | 3,144 bp linear mRNA | CCCTGTGTCTTCCCCTTCAC or SEQ | 309 |
| | | Accession: | ID NO: 52 | |
| | | NM 001302509.2 | GTCAGGAGAGGCCCCATAGA | |
| | | GI: 1890263847 | or | |
| | | | SEQ ID NO: 53 | |
| 25. | MMP-3 | 1,434 bp linear mRNA | GGTGTGGAGTTCCTGACGTT or SEQ | 286 |
| | | Accession: J03209.1 | ID NO: 54 | |
| | | GI: 188618 | GCATAGGCATGGGCCAAAAC or | |
| | | | SEQ ID NO: 55 | |
| 26. | MMP-9 | 2,336 bp linear mRNA | GCAATGCTGATGGGAAACCC | 321 |
| | | Accession: NM 004994.3 | or | |
| | | GI: 1519311730 | SEQ ID NO: 56 | |
| | | | AGAAGCCCCACTTCTTGTCG or SEQ | |
| | | | ID NO: 57 | |
| 27. | Fascin | 16,952 bp linear DNA | CTCTTTCCGTCCTTCCCGTC or SEQ | 339 |
| | | Accession: AY044229.1 | ID NO: 58 | |
| | | GI: 15625240 | CTCAGCCCCAAGACACACAC or SEQ | |
| | | | ID NO: 59 | |
| 28. | Vimentin | 1,749 bp linear DNA | CTCTGGCACGTCTTGACCTT or SEQ | 231 |
| | | Accession: M14144.1 | ID NO: 60 | |
| | | GI: 340218 | ACCATTCTTCTGCCTCCTGC or SEQ | |
| | | | ID NO: 61 | |
| 29. | RhoU | 1,619 bp linear mRNA | GCCCCTCATCCTTCCAGAAC or SEQ | 299 |
| | | Accession: AK289971.1 | ID NO: 62 | |
| | | GI: 158260964 | CTGTTGCTGAGTGTCCGAGT or SEQ | |
| | | | ID NO: 63 | |
| 30. | ICAM-1 | 1,846 bp linear mRNA | ACAACAGGCCCAAAAAGGGA or | 230 |
| | | Accession: X06990.1 | SEQ ID NO: 64 | |
| | | GI: 32614 | AGGTACCATGGCCCCAAATG or SEQ | |
| | | | ID NO: 65 | |
| 31. | NGAL | 603 bp linear mRNA | CCATGGTGCCCCTAGGTCT or SEQ | 398 |
| | | Accession: EU644752.1 | ID NO: 66 | |
| | | GI: 187370732 | CACCACTCGGACGAGGTAAC | |
| | | | or | |
| | | | SEQ ID NO: 67 | |
| 32. | POMC | 801 bp linear mRNA | GGGGTCCCACGAATCTTGTT or SEQ | 216 |
| | | Accession: CR541826.1 | ID NO: 68 | |
| | | GI: 49456608 | GTCTAGCCAAGATGGCAGTCA or | |
| | | | SEQ ID NO: 69 | |
| 33. | SAA1 | 366 bp linear mRNA | TCCTTGGCGAGGCTTTTGAT or SEQ | 250 |
| | | Accession: CR542241.1 | ID NO: 70 | |
| | | GI: 49457475 | CCACTCCTGCCCCATTCATT or SEQ | |
| | | | ID NO: 71 | |
| 34. | VEGF-A | 712 bp linear mRNA | TTCTGGGCTGTTCTCGCTTC or SEQ | 446 |
| | | Accession: BC011177.1 | ID NO: 72 | |
| | | GI: 15029904 | ACGACCGCTTACCTTGGCAT or SEQ | |
| | | | ID NO: 73 | |
| 35. | bFGF | 6,801 bp linear mRNA | AACAGGACCTGGGCAGAAAG or | 287 |
| | | Accession: NM 002006.6 | SEQ ID NO: 74 | |
| | | GI: 2198916679 | AGCTTCACTGGGTAACAGCA or SEQ | |
| | | | ID NO: 75 | |
| 36. | HGF | 2,550 bp linear mRNA | TGTCTTTGAGAAAGTACGGCAC or | 320 |
| | | Accession: FJ830862.1 | SEQ ID NO: 76 | |
| | | GI: 260542697 | AGGTGAGATGGAAAAGCTGGT or | |
| | | | SEQ ID NO: 77 | |
| 37. | AKT | 5,628 bp linear mRNA | TTGGGGATCCTCGGAGATGT or SEQ | 506 |
| | | Accession: DQ310704.1 | ID NO: 78 | |
| | | GI: 83638026 | ACAGTGACAGCCTCTGAACG or SEQ | |
| | | | ID NO: 79 | |
| 38. | PIM-1 | 2,820 bp linear mRNA | TTCATCACGGAAAGGGGAGC or | 247 |
| | | Accession: DQ022562.1 | SEQ ID NO: 80 | |
| | | GI: 68165001 | GGATCCACTCTGGAGGGCTA or SEQ | |
| | | | ID NO: 81 | |
| 39. | TNF-R2 | 3,687 bp linear mRNA | ACCGGGAGCTCAGATTCTTC or SEQ | 300 |
| | | Accession: NM 001066.3 | ID NO: 82 | |
| | | GI: 1520685528 | GCCTGGTTAACTGGGCTTCA or SEQ | |
| | | | ID NO: 83 | |
| 40. | S1P-R1 | 2,093 bp linear | TGGCTTCTCCCAGCCTATCT or SEQ | 192 |
| | | transcribed-RNA | ID NO: 84 | |
| | | Accession: NR 174350.1 | TGTGATGCTTGCAAAGTGCC or SEQ | |
| | | GI: 2178106540 | ID NO: 85 | |
| 41. | MUC-1 | 1,016 bp linear mRNA | TGCTTACAGCTACCACAGCC or SEQ | 280 |
| | | Accession: | ID NO: 86 | |
| | | NM 001204296.2 | CCAGACTGGGCAGAGAAAGG | |
| | | GI: 1677538161 | or | |
| | | | SEQ ID NO: 87 | |
| 42. | FOXO1 | 1,643 bp linear mRNA | GCAGCCGCCACATTCAACAG or SEQ | 288 |
| | | Accession: MK390615.1 | ID NO: 88 | |
| | | GI: 1725297938 | ATTTGGGGGAACGAAGCCG or SEQ | |
| | | | ID NO: 89 | |
| 43. | FOXO3A | 1,296 bp linear mRNA | GTGTTCCAGGGGAAGCACAT or SEQ | 302 |
| | | Accession: EF534714.1 | ID NO: 90 | |
| | | GI: 146262390 | GCTCTTGCCAGTTCCCTCAT or SEQ | |
| | | | ID NO: 91 | |
| 44. | Foxp3 | 3,300 bp linear DNA | CAGGCCACATTTCATGCACC or SEQ | 350 |
| | | Accession: AB007828.1 | ID NO: 92 | |
| | | GI: 2516265 | GGAAGTCCTCTGGCTCTTCG or SEQ | |
| | | | ID NO: 93 | |
| 45. | Necdin | 600 bp linear mRNA | TGATGCTCTAAGTCCACTGCC or | 317 |
| | | Accession: S67388.1 GI: | SEQ ID NO: 94 | |
| | | 453134 | TTGGGAAACGCTTACCTGGG or SEQ | |
| | | | ID NO: 95 | |
| 46. | P21 | 4,717 bp linear mRNA | AGGTCAGTTCCTTGTGGAGC or SEQ | 306 |
| | | Accession: NM 005417.5 | ID NO: 96 | |
| | | GI: 1890334261 | GAAGGTAGAGCTTGGGCAGG or | |
| | | | SEQ ID NO: 97 | |
| 47. | PI3Kp50a | 5,487 bp linear mRNA | ACACAGAGGGAGACTGGTGG or | 247 |
| | | Accession: | SEQ ID NO: 98 | |
| | | NM 001328653.2 | TTGGCGTTGTCGAAGTCAGA or SEQ | |
| | | GI: 1676319407 | ID NO: 99 | |
| 48. | PI3Kp55a | 613 bp linear DNA | CGGGATATGCCAGATGGGAC or | 247 |
| | | Accession: AF039224.1 | SEQ ID NO: 100 | |
| | | GI: 2789646 | TGGACACTGGGTACATCAGC or SEQ | |
| | | | ID NO: 101 | |

or

| | | | | |
|---|---|---|---|---|
| 49. | IL-6 | 4,011 bp linear DNA | TGCGATGGAGTCAGAGGAAAC or | 274 |
| | | Accession: MH180383.1 | SEQ ID NO: 102 | |
| | | GI: 1631959270 | GGCTAGCGCTAAGAAGCAGA | |
| | | | or | |
| | | | SEQ ID NO: 103 | |
| 50. | TNF-a | 1,211 bp linear mRNA | AAAACAACCCTCAGACGCCA | 323 |
| | | Accession: NM 000619.3 | or | |
| | | GI: 1519243198 | SEQ ID NO: 104 | |
| | | | GGAGAGTGGATGAAGGCTGG | |
| | | | or | |
| | | | SEQ ID NO: 105 | |
| 51. | IFN-Y | 309 bp linear mRNA | CAGCTCTGCATCGTTTTGGG or SEQ | 418 |
| | | Accession: AF043341.1 | ID NO: 106 | |
| | | GI: 2905631 | CTGTTTTAGCTGCTGGCGAC or SEQ | |
| | | | ID NO: 107 | |
| 52. | RANTES | 30,233 bp linear DNA | TCATTGCTACTGCCCTCTGC or SEQ | 201 |
| | | Accession: AF442818.1 | ID NO: 108 | |
| | | GI: 17933262 | TCTTCTCTGGGTTGGCACAC or SEQ | |
| | | | ID NO: 109 | |
| 53. | CRP | 2,353 bp linear mRNA | ACTTGAGTGAGTCCAAGCTGT or | 384 |
| | | Accession: GU211347.1 | SEQ ID NO: 110 | |
| | | GI: 281313276 | AGCCAGCTGCTGTCATCTTT or SEQ | |
| | | | ID NO: 111 | |
| 54. | STAT1 | 1,819 bp linear mRNA | CACAAGGTGGCAGGATGTCT or SEQ | 399 |
| | | Accession: U16997.1 | ID NO: 112 | |
| | | GI: 758419 | TCCCCGACTGAGCCTGATTA or SEQ | |
| | | | ID NO: 113 | |
| 55. | RORyt | 10,998 bp linear mRNA | CTCACGGGAGCTGCTGG or SEQ ID | 363 |
| | | Accession: NM 134260.3 | NO: 114 | |
| | | GI: 1890284460 | GGTTCCTGTTGCTGCTGTTG or SEQ | |
| | | | ID NO: 115 | |
| 56. | RORa | 913 bp linear mRNA | CCCCATACTCCTCCCCATCAT or | 364 |
| | | Accession: NM 006399.5 | SEQ ID NO: 116 | |
| | | GI: 1732746172 | CTGCCTCCAGGAACAACAGA or | |
| | | | SEQ ID NO: 117 | |
| 57. | BATF | 5,320 bp linear mRNA | TCCACCAACCTCATGTCAGC or SEQ | 306 |
| | | Accession: U52682.1 | ID NO: 118 | |
| | | GI: 1378108 | GCCATGGGACTTGAGCATCT or SEQ | |
| | | | ID NO: 119 | |
| 58. | IRF4 | 5,764 bp linear mRNA | ACCTCGCACTCTCAGTTTCAC or | 235 |
| | | Accession: NM 000565.4 | SEQ ID NO: 120 | |
| | | GI: 1519473546 | TACTTGCCGCTGTCGATCTG or SEQ | |
| | | | ID NO: 121 | |
| 59. | IL-6Ra | 2,826 bp linear mRNA | GGTCAAGGACCTCCAGCATC or SEQ | 271 |
| | | Accession: AF461422.1 | ID NO: 122 | |
| | | GI: 21239251 | AGAATCTTGCACTGGGAGGC or SEQ | |
| | | | ID NO: 123 | |
| 60. | IL-23R | 1,871 bp linear mRNA | GCCTGGCTCTGAAGTGGAAT or SEQ | 337 |
| | | Accession: NM 002190.3 | ID NO: 124 | |
| | | GI: 1393386903 | CCAAAGCCGAGCTGTTGTTT or SEQ | |
| | | | ID NO: 125 | |
| 61. | IL-17A | 813 bp linear mRNA | CCCCTAGACTCAGGCTTCCT or SEQ | 153 |
| | | Accession: NM 052872.4 | ID NO: 126 | |
| | | GI: 1714603669 | AGTTCGTTCTGCCCCATCAG or SEQ | |
| | | | ID NO: 127 | |
| 62. | IL-17F | 769 bp linear mRNA | GAAAACCAGCGCGTTTCCAT or SEQ | 393 |
| | | Accession: NM 003254.3 | ID NO: 128 | |
| | | GI: 1519313839 | GGGAATTGGGGGTCAGACAG | |
| | | | or | |
| | | | SEQ ID NO: 129 | |
| 63. | TIMP-1 | 900 bp linear mRNA | ATCCTGTTGTTGCTGTGGCT or SEQ | 229 |
| | | Accession: CR456841.1 | ID NO: 130 | |
| | | GI: 48145798 | GGGTGTAGACGAACCGGATG or | |
| | | | SEQ ID NO: 131 | |
| 64. | JunB | 3,444 bp linear mRNA | GGCTCGGTCCTGTATGTGTC or SEQ | 259 |
| | | Accession: AF051164.1 | ID NO: 132 | |
| | | GI: 2944098 | ATCGGTCCTTGTATGGGCAG or SEQ | |
| | | | ID NO: 133 | |
| 65. | iNOS | 1,452 bp linear mRNA | GCATTCAGATCCCGAAACGC or SEQ | 212 |
| | | Accession: EU812118.1 | ID NO: 134 | |
| | | GI: 209362285 | GCCCTCGAAGGTGAGTTGAA or SEQ | |
| | | | ID NO: 135 | |
| 66. | CDC25A | N.A. | AGAATGAGGAGGAGACCCCC or | 291 |
| | | | SEQ ID NO: 136 | |
| | | | TTCCTTTGGGGGAAGATGCC or SEQ | |
| | | | ID NO: 137 | |

Interestingly, 10 nM doses of both SIM1 and SIM2 strongly attenuated expressions of selective tumor-promoting factors such as AKT, PI3K-P55α, and bFGF; genes of inflammatory cytokines including IL6, RANTES, IL-17 and TGFβ; genes of metastatic proteins such as MMP1, MMP2, MMP3, MMP9, vimentin, and ICAM-1; and genes of anti-apoptotic proteins such as Cyclin D1, survivin, Bcl-2, and Bcl-XL. Therefore, both SIM1 and SIM2 could be very effective for the suppression of a diverse range of genes involved in the regression of TNBC tumors. While combining all these data and analyzing via pathway-based STRING network analysis, IL6 was identified in the primary node (Refer to FIG. 10B) of both SIM1- and SIM2-regulated genes suggesting that both SIM1- and SIM2-mediated suppression IL6 gene could be a critical factor in the apoptosis of TNBC cells. Third, to further confirm the promoter recruitment activity of Y705PSTAT3 in the IL6 promoter, chromatin immunoprecipitation assay of STAT3-response element in the IL6 promoter was performed using primer sets described under “Promoter analysis and Chromatin Immunoprecipitation (ChIP) assay” hereinabove. Briefly, MDA-MB-468 cells were incubated with 10 ng/mL EGF, EGF+SIM-1 (20 nM), and EGF+SIM-2 (20 nM) for 5 hrs followed by performing an immunoprecipitation (IP) of DNA-bound STAT3 with Y705P STAT3-specific antibody and then the precipitated DNA by PCR analysis was analyzed to identify STAT3 response element (˜121 bp long) in IL6 promoter. The analysis (FIG. 9C) revealed that EGF significantly induced the recruitment of Y705P STAT3 in the STAT3 response element of the IL6 promoter and both SIM-1 and SIM-2 significantly attenuated the promoter recruitment activity of Y70P STAT3.

In fact, current litterateurs [51; 52] suggest that TNBC cells secrete a strong level of IL6 [53] that promotes the proliferation of TNBC cells by autocrine mode via stimulating IL6 receptors expressed on TNBC cells. The data is aligned with the ChIP study that demonstrated how both SIM1 and SIM2 suppressed the binding of STAT3 in the promoter of IL6. Once recruited to the DNA, STAT3 controls the gene transcription. Therefore finally, an eGFP reporter assay was performed to evaluate if SIM-1 and SIM-2 inhibited the transcriptional activity of STAT3. An eGFP reporter cassette was cloned under the guidance of IL6 promoter carrying STAT response element (FIG. 10C), transfected in MDA-MB-468 cells, and then treated by EGF (20 ng/mL) along with different doses of SIM-1 and SIM-2. Interestingly, both SIM-1 (FIG. 10D) and SIM-2 (FIG. 10E) dose-dependently inhibited the EGF-induced GFP reporter activity in MDA-MB-468 cells suggesting that both SIM-1 and SIM-2 inhibited the transcriptional activity of STAT3. Moreover, the site-directed mutation of the STAT3 response element (FIG. 10F) following similar transfection studies revealed that both SIM-1 and SIM-2 failed to induce the GFP reporter activity in EGF-treated cells (FIG. 10G) reiterating the STAT3 inhibitory role of both SIM-1 and SIM-2.

Once the transcriptional regulation of STAT3-dependent genes was confirmed, the quantification of IL6 was performed. A real-time PCR analysis of the IL6 gene (FIG. 10H) in MDA-MB-468 cells after treating 20 nM of SIM-1 and SIM-2 for 5 hrs. Intriguingly, both SIM-1 and SIM-2 strongly inhibited the gene expression of IL6 in MDA-MB-468 cells. Next, the suppression of IL6 by ELISA analysis (FIG. 10I) was confirmed. ELISA was performed in the supernatants of MDA-MB-468 cells after stimulation with EGF followed by treating dose-dependently with SIM-1 and SIM-2 for 48 hrs. Similarly, the gene (FIG. 10J) and protein (FIG. 10K) expressions of RANTES, a STAT3-dependent tumor promoting-chemokine [54; 55], were also found to be significantly downregulated at 20 nM doses of SIM-1 and SIM-2. Collectively, the results of this example provide that although both SIM-1 and SIM-2 effectively downregulated the expressions of a diverse range of STAT3-dependent tumor-promoting genes, the downregulation of IL6 could play a central role in the SIM-1- and SIM-2-mediated suppression of all other tumor-promoting factors.

### Example 4: SIMMPYRAs Promote Apoptosis of TNBC Cells, not Healthy Human Mammary Epithelial Cells

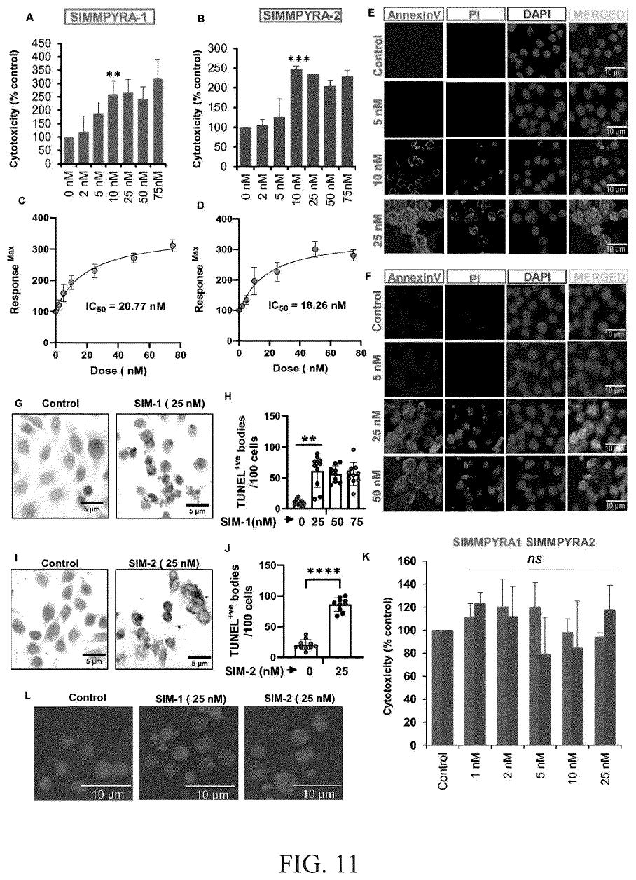

This example provides a representative experiment that studies if SIM-1 and SIM-2 promoted the death of TNBC cells. The following experiments were adopted to confirm the apoptosis of TNBC cells. First, a colorimetric LDH (lactate dehydrogenase) release assay was performed in the supernatants of both SIM-1- (FIG. 11A) and SIM-2- (FIG. 11B) treated MDA-MB-468 cells. A dose dependent study confirmed that both SIM-1 and SIM-2 promoted death of these TNBC cells with increasing doses reaching maximum at 20 nM concentrations. Second, in order to determine the optimum dose of cytotoxicity, CT50 (half-response of cytotoxicity) analysis was performed by plotting maximum response (arbitrary unit) as an index of dose. Accordingly, that analysis revealed that both SIM-1 and SIM-2 promoted cytotoxicity at CT50=20.77 (FIG. 11C) and 18.26 nM (FIG. 11D) concentrations respectively. Third, we performed annexin V and propidium iodide dual labeling in order to understand the apoptosis of TNBC cells at the molecular level as described hereinabove. During apoptosis, phosphatidyl serine of the plasma membrane flips from the inner to the outer membrane which can be detected by Annexin V labeling, whereas PI stains the nucleus of a cell with compromised integrity of the plasma membrane. Therefore, dual staining of Annexin V and PI confirms the cellular changes of apoptosis. Accordingly, we observed that both SIM-1 (FIG. 11E) and SIM-2 (FIG. 11F) dose-dependently increased the membrane labeling of Annexin V and nuclear staining of PI with maximum at 25 nM concentrations confirming the apoptosis-inducing effects of SIMMPYRAs. Finally, a TUNEL assay was performed, which is considered a gold-standard technique to identify the hallmark nuclear disintegration phenomenon of apoptosis. Interestingly, 25 nM doses of both SIM-1 (FIG. 11G and FIG. 11H) and SIM-2 (FIG. 1I and FIG. 11J) strongly induced TUNEL response in MDA-MB-468 cells as indicated by brown TUNEL-positive bodies in the background of cresyl violet-stained blue nuclei of cells. The result was further confirmed by quantifying the total numbers of TUNEL-positive cells per 100 total cells per group as demonstrated by histogram analyses (FIG. 11H and FIG. 11J).

To nullify the off-target cell death, we performed an LDH assay in the healthy human mammary epithelial cells (HMECs). Interestingly, 1, 2, 5, 10, and nM doses of both SIM-1 and SIM-2 (FIG. 11K) were unable to augment any significant cell death in HMEC cells compared to DMSO control. Nevertheless, our annexin V/PI staining (FIG. 11L) further reiterated that both SIM-1 and SIM-2 were indeed unable to induce apoptosis in healthy normal cells.

### Example 5: SIMMPYRAS Arrest Growth and Promote Apoptosis of TNBC Tumors in PDX Mouse Model

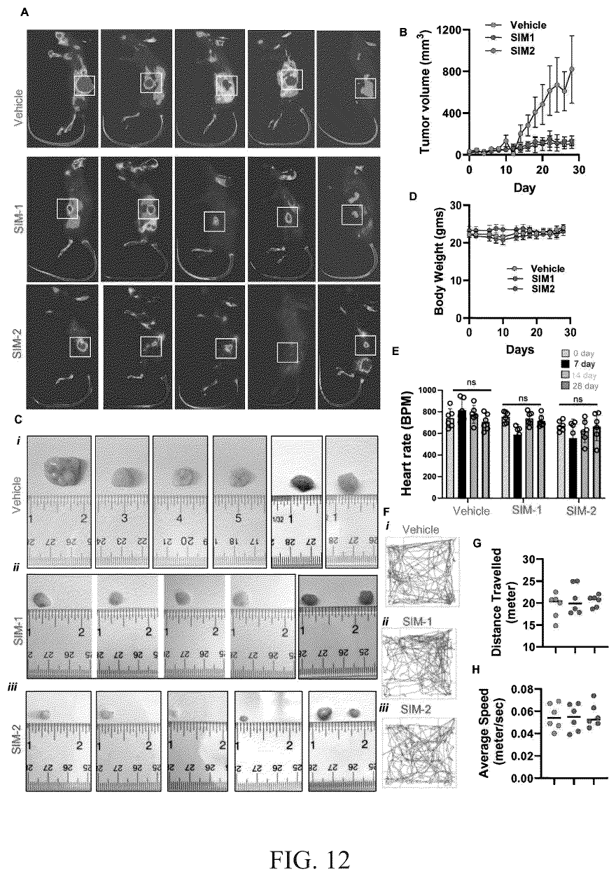

This example provides a representative experiment of the present invention that studies the effects of both SIM-1 and SIM-2 on the regression and growth arrest of tumors in vivo in the PDX mouse model of TNBC. Development of tumors, the housing of animals, drug solubilization, treatment regimen, health follow-up, and endpoint analyses had been performed according to an approved IACUC protocol and discussed in detail hereinabove. Briefly, once tumor size reached approximately 4 mm in size (both length and width), vehicle, SIM-1, and SIM-2 were administered at a dose of 2 mg/Kg Bwt via intraperitoneal or i.p. route for two weeks at every alternate day. After two weeks, the drug administration was stopped, but animals were monitored daily for another two weeks to verify if the tumor grew back due to the withdrawal of the drug. During that entire study period, tumor size was recorded by a digital Vernier Caliper, and health vitals such as heart rate, blood pressure, and oxygen saturation were monitored by MouseStatJr (Kent Scientific). At the end of the 4th week, IRDye680-tagged 2 DG dye was administered via the tail vein, waited overnight, and then imaged in a Licor infrared scanner. According to the image analysis (FIG. 12A), Tumors grew aggressively in the vehicle-treated group, whereas both SIM-1 and SIM-2 significantly arrested the growth of the tumor (FIG. 12A). While measuring the tumor volume (FIG. 12B), the vehicle-treated group displayed a strong rise in the growth curve over a month starting from an initial volume of 30.14±26.75 mm³ (mean±SD) recorded at day 0 to the final volume=949.15±769.15 mm³ (***p<0.005=0.00315 vs. day 0). Interestingly, both SIM-1 and SIM-2 consistently suppressed the growth of the tumor starting from an initial volume of 37.65±15.41 and 37.43±7.07 mm³ to the final volume of 126.69±29.98 and 134.09±41.77 mm³ (*p<0.05 vs. day 0; marginally significant) respectively.

At the end of the study, tumors were carefully harvested from the flank and the endpoint sizes were displayed along with a ruler as shown in FIG. 12C. During the entire study period, mice did not display any loss in body weight (FIG. 12D) and heart rate (FIG. 12E) across all different treatment groups. In order to assess the effects of drugs on the movement of mice, next we performed open open-field test. Accordingly, the gross movement as shown by trackplot analysis (FIG. 12F), total distance traveled (FIG. 12G), and average speed (FIG. 12H) measurements did not show any adverse outcome due to the treatments of SIM-1 and SIM-2. Collectively, these results suggest that both SIM-1 and SIM-2 strongly suppressed the growth of human TNBC tumors.

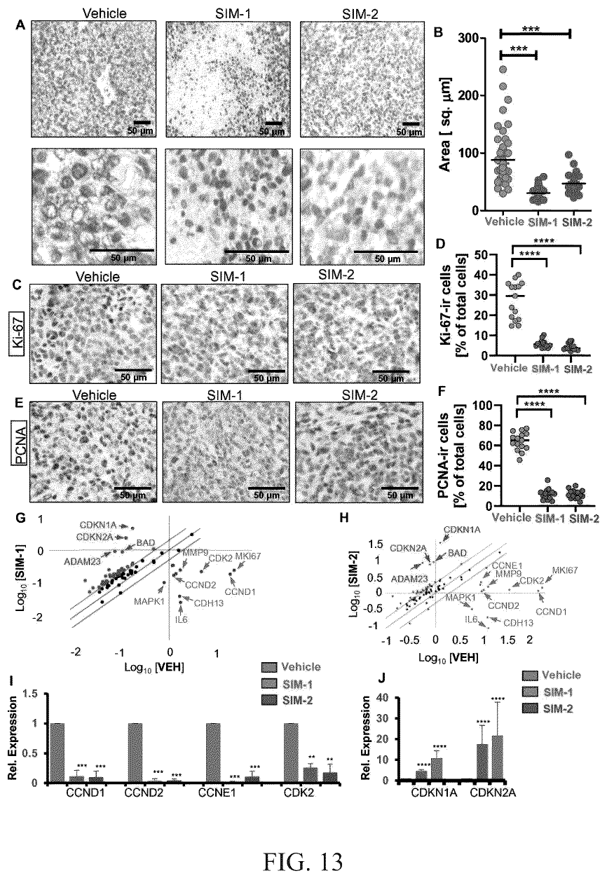

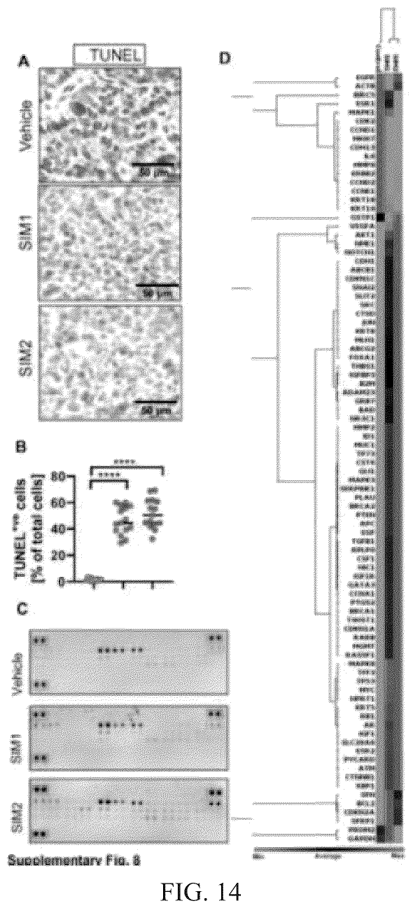

To assess the histopathological changes in tumor parenchyma, H&E staining of tumor tissue was performed in three different groups (FIG. 13A). The hematoxylin-stained cells appeared with enlarged and densely colored nuclei characteristic of growing and mitotic tumor cells. On the contrary, both SIM-1 and SIM-2 treatments strongly reduced the size and color intensities of cells suggesting that both these compounds may display growth-suppressive effects in tumors. The result was further corroborated with the quantitative analyses of cell size (FIG. 13B). To further demonstrate the effect of drugs on the suppression of tumor growth, IHC analyses with cell growth marker Ki-67 (kiel-67) (FIG. 13C) and cell division marker PCNA (proliferative cell nuclear antigen) (FIG. 13E) were performed. Interestingly, Ki-67 staining followed by the quantitative analysis (FIG. 13D) displayed that both SIM-1 and SIM-2 displayed a strong reduction of Ki-67 immunoreactivity in the tumor parenchyma (***p<0.0001 vs control as determined by one-way ANOVA with treatment as a single effector). Similarly, while analyzing the expression cell proliferation marker PCNA, it was observed that treatments of both SIMs strongly suppressed PCNA immunoreactivity (*p<0.0001 vs. control; One-way ANOVA) (FIG. 13F). These results collectively suggest that SIMMPYRAs effectively suppressed the growth of TNBC tumors. To further confirm, we performed a gene array with a validated RNA array kit (Qiagen) of genes that are involved in the growth and proliferation of breast cancer cells. Relative expressions of different genes were summarized by heatmap clusterogram analyses (FIG. 14D). The Qiagen data analysis server-derived scatterplot comparison of logarithmic expressions of genes between vehicle and SIM-1 (FIG. 13G) and between vehicle and SIM-2 (FIG. 13I) demonstrated upregulated genes as red dots in the upper left quadrant and downregulated genes as green dots in the lower right quadrant of the graphs. These analyses clearly indicated that the majority of altered genes regulate cell cycle progression. The upregulated genes such as CDKN1A (Cyclin Dependent Kinase Inhibitor 1A) and CDKN2A (Cyclin Dependent Kinase Inhibitor 1A) inhibit cell cycle, whereas downregulated genes such as CCND1 (Cyclin D1), CCND2 (Cyclin D2), CCNE1 (Cyclin E1), and CDK2 (Cyclin Dependent Kinase 2) stimulate cell cycle progression. Even though the TUNEL assay (FIG. 14A) followed by quantification analysis (FIG. 14B) indicated strong apoptosis, neither the validated protein array of apoptotic markers (FIG. 14C) nor the gene array (FIG. 14D) showed any significant changes in apoptotic markers.

Subsequently, real-time PCR analyses of upregulated genes (FIG. 13I) clearly demonstrated that genes upregulating cell division and cell cycle progression such as CCND1 (Cyclin D1), CCND2 (Cyclin D2), CCNE1 (Cyclin E1), and CDK2 (Cyclin Dependent Kinase 2) were strongly suppressed after SIM-1 and -2 treatments. On the contrary, genes inhibiting cell cycle progression such as CDKN1A (Cyclin Dependent Kinase Inhibitor 1A) and CDKN2 Å (Cyclin Dependent Kinase Inhibitor 1A) were found to be significantly induced (FIG. 13J) after SIM-1 and -2 treatments. Therefore, the results of the present example show that upon treatments with SIM-1 and SIM-2, the growth-suppressive effect surpassed the apoptotic response in these TNBC tumors.

### Example 6: SIM-1 and 2 Ameliorate Y705 Phosphorylation of STAT3 and Suppress the Expressions of Tumor Promoting Genes in TNBC Tumor Tissue

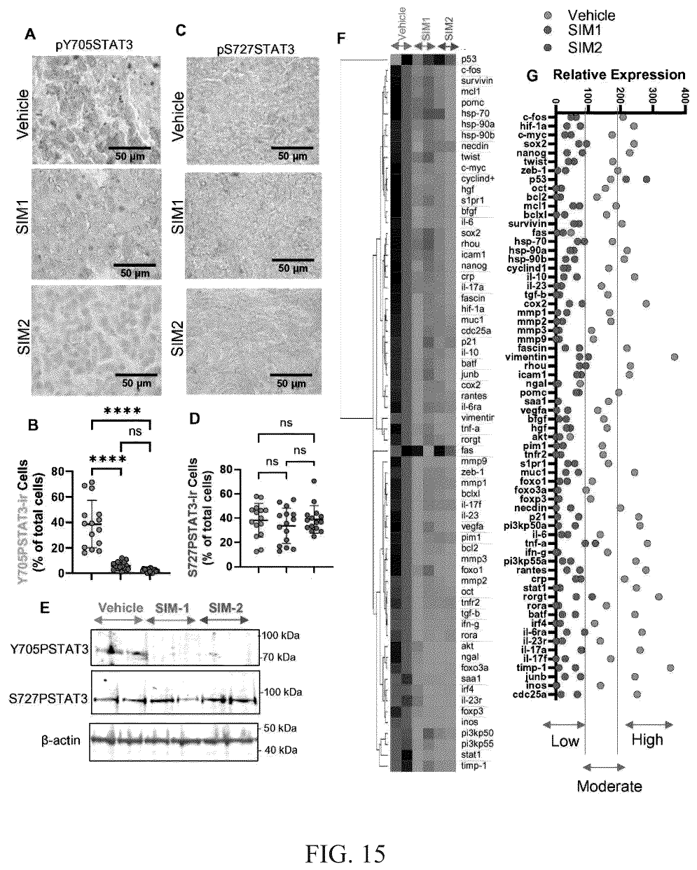

This example provides a representative experiment that tested if SIM-1 and SIM-2 suppressed the activation of STAT3 in TNBC tumors. At first, an IHC analysis of Y705P STAT3 revealed that vehicle-treated TNBC tumors strongly expressed Y705P STAT3 in the nuclei of enlarged and proliferating tumor cells (FIG. 15A). Interestingly, SIM-1 treatment significantly suppressed (FIG. 15A; 2^{nd} panel) and SIM-2 treatment completely ameliorated (FIG. 15A; 3^{rd} panel) the expression of Y705PSTAT3. The result was further corroborated by the quantitative analysis (FIG. 15B), which indicated a significant downregulation of Y705PSTST3-ir cells (One-way ANOVA indicated ****p<0.0001 versus vehicle).

On the contrary, the expression of S727PSTAT3, another active isomer of STAT3, remained very low and unchanged across the treatment groups (FIG. 15C). The quantitative analyses (FIG. 15D) further confirmed that both SIM-1 and SIM-2 did not show any change in S727P STAT3 expressions in TNBC tumors.

Next, the immunoblot analyses (FIG. 15E) of Y705P STAT3 and S727P STAT3 further demonstrated that both SIM-1 and SIM-2 strongly and selectively suppressed the Y705 phosphorylation of STAT3, but not S727PSTAT3. Then, a comprehensive gene analysis of 66 STAT3-dependent, tumor-promoting genes (FIG. 15F) was performed as indicated elsewhere [41] following a scatter plot analysis (FIG. 15G), which summarized the expressions of all genes with a total of 55 out of 66 genes strongly downregulated. These genes include anti-apoptotic genes such as bcl-2, survivin, and bcl-xl; cell cycle progression markers such as cyclin D1, akt, and p21. Interestingly, tumor growth suppressor gene p53 was found to be significantly upregulated after SIM-1 and SIM-2 treatments. Combinedly, the results of this example show that both SIM-1 and SIM-2 bind to STAT-3, strongly attenuate the expression of Tyr 705 phosphorylated STAT3, suppress the expressions of tumor-promoting genes, and strongly promote the growth arrest of TNBC tumors.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

### REFERENCES

[1] W. J. Irvin, Jr., and L. A. Carey, What is triple-negative breast cancer?Eur J Cancer 44 (2008) 2799-805.
[2] J. M. Dolle, J. R. Daling, E. White, L. A. Brinton, D. R. Doody, P. L. Porter, and K. E. Malone, Risk factors for triple-negative breast cancer in women under the age of 45 years. Cancer Epidemiology Biomarkers & Prevention 18 (2009) 1157-1166.
[3] T. Ovcaricek, S. Frkovic, E. Matos, B. Mozina, and S. Borstnar, Triple negative breast cancer-prognostic factors and survival. Radiology and oncology (2011) 46-52.
[4] C. Liedtke, K. Hess, T. Karn, A. Rody, L. Kiesel, G. Hortobagyi, L. Pusztai, and A. Gonzalez-Angulo, The prognostic impact of age in patients with triple-negative breast cancer. Breast cancer research and treatment 138 (2013) 591-599.
[5] C. K. Anders, L. A. Carey, and H. J. Burstein, ER/PR negative, HER2-negative (triple-negative) breast cancer. UpToDate; Vora, SR, Ed.; Wolters Kluwer: Waltham, MA, USA (2021).
[6] V. C. Jordan, and B. W. O'Malley, Selective estrogen-receptor modulators and antihormonal resistance in breast cancer. J Clin Oncol 25 (2007) 5815-24.
[7] J. Liu, Tamoxifen metabolites can target both aromatase and estrogen receptors. (2015).
[8] J. F. Bromberg, M. H. Wrzeszczynska, G. Devgan, Y. Zhao, R. G. Pestell, C. Albanese, and J. E. Darnell, Jr., Stat3 as an oncogene. Cell 98 (1999) 295-303.
[9] J.-J. Qin, L. Yan, J. Zhang, and W.-D. Zhang, STAT3 as a potential therapeutic target in triple negative breast cancer: a systematic review. Journal of Experimental & Clinical Cancer Research 38 (2019) 1-16.
[10] A. Xiong, Z. Yang, Y. Shen, J. Zhou, and Q. Shen, Transcription Factor STAT3 as a Novel Molecular Target for Cancer Prevention. Cancers (Basel) 6 (2014) 926-57.
[11] G. Chalikonda, H. Lee, A. Sheik, and Y. S. Huh, Targeting key transcriptional factor STAT3 in colorectal cancer. Mol Cell Biochem 476 (2021) 3219-3228.
[12] N. Don-Doncow, Z. Escobar, M. Johansson, S. Kjellstrom, V. Garcia, E. Munoz, O. Sterner, A. Bjartell, and R. Hellsten, Galiellalactone is a direct inhibitor of the transcription factor STAT3 in prostate cancer cells. J Biol Chem 289 (2014) 15969-78.
[13] J. E. Darnell, Validating Stat3 in cancer therapy. Nat Med 11 (2005) 595-6.
[14] T. Ashizawa, H. Miyata, H. Ishii, C. Oshita, K. Matsuno, Y. Masuda, T. Furuya, T. Okawara, M. Otsuka, N. Ogo, A. Asai, and Y. Akiyama, Antitumor activity of a novel small molecule STAT3 inhibitor against a human lymphoma cell line with high STAT3 activation. Int J Oncol 38 (2011) 1245-52.
[15] A. N. Gargalionis, K. A. Papavassiliou, and A. G. Papavassiliou, Targeting STAT3 Signaling Pathway in Colorectal Cancer. Biomedicines 9 (2021).
[16] Z. Wang, J. Li, W. Xiao, J. Long, and H. Zhang, The STAT3 inhibitor S31-201 suppresses fibrogenesis and angiogenesis in liver fibrosis. Lab Invest 98 (2018) 1600-1613.
[17] M. Liang, F. Zhan, J. Zhao, Q. Li, J. Wuyang, G. Mu, D. Li, Y. Zhang, and X. Huang, CPA-7 influences immune profile and elicits anti-prostate cancer effects by inhibiting activated STAT3. BMC Cancer 16 (2016) 504.
[18] J. Schust, B. Sperl, A. Hollis, T. U. Mayer, and T. Berg, Stattic: a small-molecule inhibitor of STAT3 activation and dimerization. Chem Biol 13 (2006) 1235-42.
[19] Bai, L., Zhou, H., Xu, R., Zhao, Y., Chinnaswamy, K., McEachern, D. et al. (2019) A Potent and Selective Small-Molecule Degrader of STAT3 Achieves Complete Tumor Regression In Vivo Cancer Cell 36, 498-511 e417 10.1016/j.ccell.2019.10.002.
[20] W. Li, H. Yang, X. Li, L. Han, N. Xu, and A. Shi, Signaling pathway inhibitors target breast cancer stem cells in triple-negative breast cancer. Oncol Rep 41 (2019) 437-446.
[21] Levy, D. E., andLee, C. K. (2002) What does Stat3 do?J Clin Invest 109, 1143-1148 10.1172/JC115650.
[22] Hong, X. Y., Wan, H. L., Li, T., Zhang, B. G., Li, X. G., Wang, X. et al. (2020) STAT3 ameliorates cognitive deficits by positively regulating the expression of NMDARs in a mouse model of FTDP-17 Signal Transduct Target Ther 5, 295 10.1038/s41392-020-00290-9.
[23] A. Roy, M. Kundu, S. Chakrabarti, D. R. Patel, and K. Pahan, Oleamide, a Sleep-Inducing Supplement, Upregulates Doublecortin in Hippocampal Progenitor Cells via PPARalpha. J Alzheimers Dis 84 (2021) 1747-1762.
[24] G. Gottschalk, D. Peterson, K. Knox, M. Maynard, R. J. Whelan, and A. Roy, Elevated ΔTG13 in serum of patients with ME/CFS stimulates oxidative stress response in microglial cells via activation of receptor for advanced glycation end products (RAGE). Mol Cell Neurosci 120 (2022) 103731.
[25] Galvao, J., Davis, B., Tilley, M., Normando, E., Duchen, M. R., andCordeiro, M. F. (2014) Unexpected low-dose toxicity of the universal solvent DMSO FASEB J 28, 1317-1330 10.1096/fj.13-235440.
[26] Lin, G. J., Wu, C. H., Yu, C. C., Lin, J. R., Liu, X. D., Chen, Y. W. et al. (2019) Adoptive transfer of DMSO-induced regulatory T cells exhibits a similar preventive effect compared to an in vivo DMSO treatment for chemical-induced experimental encapsulating peritoneal sclerosis in mice Toxicol Appl Pharmacol 378, 114641 10.1016/j.taap.2019.114641.
[27] J. J. Qin, L. Yan, J. Zhang, and W. D. Zhang, STAT3 as a potential therapeutic target in triple negative breast cancer: a systematic review. J Exp Clin Cancer Res 38 (2019) 195.
[28] P. Maycotte, C. M. Gearheart, R. Barnard, S. Aryal, J. M. Mulcahy Levy, S. P. Fosmire, R. J. Hansen, M. J. Morgan, C. C. Porter, D. L. Gustafson, and A. Thorburn, STAT3-mediated autophagy dependence identifies subtypes of breast cancer where autophagy inhibition can be efficacious. Cancer Res 74 (2014) 2579-90.
[29] L. Lu, J. Dong, L. Wang, Q. Xia, D. Zhang, H. Kim, T. Yin, S. Fan, and Q. Shen, Activation of STAT3 and Bcl-2 and reduction of reactive oxygen species (ROS) promote radioresistance in breast cancer and overcome of radioresistance with niclosamide. Oncogene 37 (2018) 5292-5304.
[30] M. Jiang, J. Chen, W. Zhang, R. Zhang, Y. Ye, P. Liu, W. Yu, F. Wei, X. Ren, and J. Yu, Interleukin-6 Trans-Signaling Pathway Promotes Immunosuppressive Myeloid-Derived Suppressor Cells via Suppression of Suppressor of Cytokine Signaling 3 in Breast Cancer. Front Immunol 8 (2017) 1840.
[31] Y. Aoki, G. M. Feldman, and G. Tosato, Inhibition of STAT3 signaling induces apoptosis and decreases survivin expression in primary effusion lymphoma. Blood 101 (2003) 1535-42.
[32] M. Gallo, D. Frezzetti, C. Roma, N. Chicchinelli, A. Barbieri, C. Arra, G. Scognamiglio, G. Botti, A. De Luca, and N. Normanno, RANTES and IL-6 cooperate in inducing a more aggressive phenotype in breast cancer cells. Oncotarget 9 (2018) 17543-17553.
[33] S. Yoon, S. U. Woo, J. H. Kang, K. Kim, M. H. Kwon, S. Park, H. J. Shin, H. S. Gwak, and Y. J. Chwae, STAT3 transcriptional factor activated by reactive oxygen species induces IL6 in starvation-induced autophagy of cancer cells. Autophagy 6 (2010) 1125-38.
[34] Y. Zheng, S. Li, H. Tang, X. Meng, and Q. Zheng, Molecular mechanisms of immunotherapy resistance in triple-negative breast cancer. Front Immunol 14 (2023) 1153990.
[35] H. Q. Wang, Q. W. Man, F. Y. Huo, X. Gao, H. Lin, S. R. Li, J. Wang, F. C. Su, L. Cai, Y. Shi, B. Liu, and L. L. Bu, STAT3 pathway in cancers: Past, present, and future. MedComm (2020) 3 (2022) e124.
[36] J. Abdulghani, L. Gu, A. Dagvadorj, J. Lutz, B. Leiby, G. Bonuccelli, M. P. Lisanti, T. Zellweger, K. Alanen, T. Mirtti, T. Visakorpi, L. Bubendorf, and M. T. Nevalainen, Stat3 promotes metastatic progression of prostate cancer. Am J Pathol 172 (2008) 1717-28.
[37] G. He, and M. Karin, NF-kappaB and STAT3-key players in liver inflammation and cancer. Cell Res 21 (2011) 159-68.
[38] R. B. Luwor, S. S. Stylli, and A. H. Kaye, The role of Stat3 in glioblastoma multiforme. J Clin Neurosci 20 (2013) 907-11.
[39] M. D. Hadjidaniel, S. Muthugounder, L. T. Hung, M. A. Sheard, S. Shirinbak, R. Y. Chan, R. Nakata, L. Borriello, J. Malvar, R. J. Kennedy, H. Iwakura, T. Akamizu, R. Sposto, H. Shimada, Y. A. DeClerck, and S. Asgharzadeh, Tumor-associated macrophages promote neuroblastoma via STAT3 phosphorylation and up-regulation of c-MYC. Oncotarget 8 (2017) 91516-91529.
[40] J. Munoz, N. Dhillon, F. Janku, S. S. Watowich, and D. S. Hong, STAT3 inhibitors: finding a home in lymphoma and leukemia. Oncologist 19 (2014) 536-44.
[41] F. Zhu, K. B. Wang, and L. Rui, STAT3 Activation and Oncogenesis in Lymphoma. Cancers (Basel) 12 (2019).
[42] P. S. Y. Chong, W. J. Chng, and S. de Mel, STAT3: A Promising Therapeutic Target in Multiple Myeloma. Cancers (Basel) 11 (2019).
[43] P. Dutta, N. Sabri, J. Li, and W. X. Li, Role of STAT3 in lung cancer. JAKSTAT 3 (2014) e999503.
[44] M. B. VanElzakker, S. A. Brumfield, and P. S. Lara Mejia, Neuroinflammation and Cytokines in Myalgic Encephalomyelitis/Chronic Fatigue Syndrome (ME/CFS): A Critical Review of Research Methods. Front Neurol 9 (2018) 1033.
[45] D. Peterson, E. W. Brenu, G. Gottschalk, S. Ramos, T. Nguyen, D. Staines, and S. Marshall-Gradisnik, Cytokines in the cerebrospinal fluids of patients with chronic fatigue syndrome/myalgic encephalomyelitis. Mediators Inflamm 2015 (2015) 929720.
[46] V. Trinh Thi Thanh, H. Doan Thi Mai, V. C. Pham, M. Litaudon, V. Dumontet, F. Gueritte, V. H. Nguyen, and V. M. Chau, Acetylcholinesterase inhibitors from the leaves of Macaranga kurzii. J Nat Prod 75 (2012) 2012-5.
[47] B. Dahlen, E. Nizankowska, A. Szczeklik, O. Zetterstrom, G. Bochenek, M. Kumlin, L. Mastalerz, G. Pinis, L. J. Swanson, T. I. Boodhoo, S. Wright, L. M. Dube, and S. E. Dahlen, Benefits from adding the 5-lipoxygenase inhibitor zileuton to conventional therapy in aspirin-intolerant asthmatics. Am J Respir Crit Care Med 157 (1998) 1187-94.
[48] A. Roy, M. Jana, M. Kundu, G. T. Corbett, S. B. Rangaswamy, R. K. Mishra, C. H. Luan, F. J. Gonzalez, and K. Pahan, HMG-CoA Reductase Inhibitors Bind to PPARalpha to Upregulate Neurotrophin Expression in the Brain and Improve Memory in Mice. Cell Metab 22 (2015) 253-65.
[49] Lim, S. O., Li, C. W., Xia, W., Lee, H. H., Chang, S. S., Shen, J. et al. (2016) EGFR Signaling Enhances Aerobic Glycolysis in Triple-Negative Breast Cancer Cells to Promote Tumor Growth and Immune Escape Cancer Res 76, 1284-1296 10.1158/0008-5472.CAN-15-2478.
[50] Carpenter, R. L., andLo, H. W. (2014) STAT3 Target Genes Relevant to Human Cancers Cancers (Basel) 6, 897-925 10.3390/cancers6020897.
[51] L. Vecchi, S. T. S. Mota, M. A. P. Zoia, I. C. Martins, J. B. de Souza, T. G. Santos, A. O. Beserra, V. P. de Andrade, L. R. Goulart, and T. G. Araujo, Interleukin-6 Signaling in Triple Negative Breast Cancer Cells Elicits the Annexin A1/Formyl Peptide Receptor 1 Axis and Affects the Tumor Microenvironment. Cells 11 (2022).
[52] Z. C. Hartman, G. M. Poage, P. den Hollander, A. Tsimelzon, J. Hill, N. Panupinthu, Y. Zhang, A. Mazumdar, S. G. Hilsenbeck, G. B. Mills, and P. H. Brown, Growth of triple-negative breast cancer cells relies upon coordinate autocrine expression of the proinflammatory cytokines IL-6 and IL-8. Cancer Res 73 (2013) 3470-80.
[53] Masjedi, A., Hashemi, V., Hojjat-Farsangi, M., Ghalamfarsa, G., Azizi, G., Yousefi, M. et al. (2018) The significant role of interleukin-6 and its signaling pathway in the immunopathogenesis and treatment of breast cancer Biomed Pharmacother 108, 1415-1424 10.1016/j.biopha.2018.09.177.
[54] Roy, A., Mondal, S., Kordower, J. H., andPahan, K. (2015) Attenuation of microglial RANTES by NEMO-binding domain peptide inhibits the infiltration of CD8(+) T cells in the nigra of hemiparkinsonian monkey Neuroscience 302, 36-46 10.1016/j.neuroscience.2015.03.011.
[55] Kovacic, J. C., Gupta, R., Lee, A. C., Ma, M., Fang, F., Tolbert, C. N. et al. (2010) Stat3-dependent acute Rantes production in vascular smooth muscle cells modulates inflammation following arterial injury in mice J Clin Invest 120, 303-314 10.1172/JCI40364.

What is claimed is:

1. A compound having Formula (I), or an isomer, a structural analog, a chemical analog, or a pharmaceutically acceptable salt thereof: 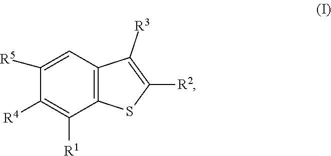
wherein R¹ is selected from a group consisting of —Cl, and —H,
wherein R² is selected from a group consisting of 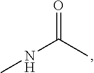
and —H,
wherein R³ is selected from a group consisting of 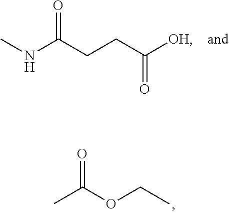
wherein R⁴ is selected from a group consisting of 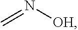
and —H, and
wherein R⁵ is selected from a group consisting of —Cl, and —H.

2. The compound of claim 1 having Formula (II), or an isomer, a structural analog, a chemical analog, or a pharmaceutically acceptable salt thereof:

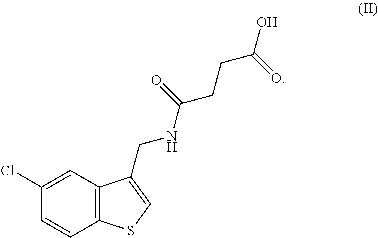 [SMILES: O=C(O)CCC(=O)NCc1csc2ccc(Cl)cc12]

3. The compound of claim 1 having Formula (III), or an isomer, a structural analog, a chemical analog, or a pharmaceutically acceptable salt thereof:

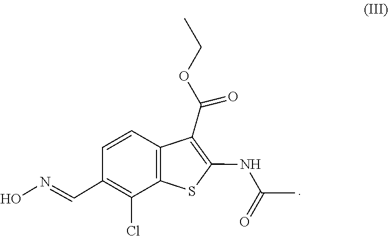 [SMILES: CCOC(=O)c1c(NC(C)=O)sc2c(Cl)c(/C=N/O)ccc12]

4. The compound of claim 1, wherein the compound is a small-molecule inhibitor targeting signal transducer and activator of transcription 3 (STAT3) that inhibits only STAT3 at a nanomolar concentration not other isoforms of signal transducer and activator of transcription factors (STATs), and wherein the compound stimulates cytotoxicity and induces apoptosis of cells with irregular STAT3 activation at nanomolar concentration, does not cause off-target side effects, and prevents STAT3 activation-driven pathogenesis.

5. The compound of claim 1, wherein the compound is comprised in a pharmaceutical composition, wherein the pharmaceutical composition comprises the compound of claim 1; and at least one of pharmaceutically acceptable carriers; excipients; diluents; adjuvants; and vehicles, wherein the pharmaceutical composition further comprises an additional therapeutic agent, wherein the additional therapeutic agent is a chemotherapeutic drug, an antiproliferative agent, an immunosuppressor, an immunologic stimulant, an anti-inflammatory reagent, or a combination thereof, wherein the compound is a small-molecule inhibitor targeting signal transducer and activator of transcription 3 (STAT3) that inhibits only STAT3 at a nanomolar concentration not other isoforms of signal transducer and activator of transcription factors (STATs), and wherein the compound stimulates cytotoxicity and induces apoptosis of cells with irregular STAT3 activation at nanomolar concentration, does not cause off-target side effects, and prevents STAT3 activation-driven pathogenesis.

6. A method of obtaining a compound of claim 1, the method comprising the steps of: (a) preparing a series of structurally related compounds having a benzothiophene backbone by extracting liner sequence of each compound in simplified molecular-input line-entry system (SMILES) format, and then converting them to three-dimensional Mol2 format with the help of Python-based algorithm; (b) screening the compounds of step (a) for their role as small-molecule inhibitors of signal transducer and activator of transcription 3 (STAT3) by employing an in-silico technique to assess binding of said compounds in the ligand binding pocket of STAT3 protein based on their docking with STAT3 in Swiss-Dock server; (c) identifying the most potent and efficient inhibitors of STAT3 from amongst the compounds of step (b) based on free-energy change and full-fitness energy ranking to identify the compound of claim 1; (d) synthesizing, and purifying the identified compound of claim 1 of step (c); (e) structurally evaluating the synthesized and purified compound of step (d) by LCMS and NMR spectroscopy; (f) assessing the compound of step (e) by biochemical and cellular assays for selective binding and molecular interaction with STAT3 at a nanomolar concentration; (g) validating functional and selective inhibition of STAT3 activation at a nanomolar concentration not other isoforms of signal transducer and activator of transcription factors (STATs) in cell-based assays by the compound of step (f), wherein steps (a) to (g) result in obtaining the compound of claim 1 as a potent and efficient selective inhibitor of STAT3 at a nanomolar concentration.

7. The method of claim 6, wherein the biochemical and cellular assays include protein thermal shift (PTS) assay, fluorescence polarization analyses, high-throughput STAT binding protein array assay, immunoblot assay, 6× luciferase reporter gene assay array for selective STAT3 selective binding and molecular interaction assessment at a nanomolar concentration of the compound of claim 1.

8. The method of claim 6, wherein the cell-based assays include assessment of specific STAT3 activation inhibition targeting suppression of phosphorylation at tyrosine 705 (Y705) of STAT3 protein, Lactate dehydrogenase cell cytotoxicity assay, dose-response analysis for apoptosis in cell-based assays, annexin-V/propidium iodide dual labeling assay, TUNEL staining assay, and apoptotic gene array assay at a nanomolar concentration of the compound of claim 1.

9. A method of preventing or treating a disorder or disease in a subject, the method comprising the steps of: (i) obtaining the compound of claim 1; (ii) preparing a pharmaceutical composition comprising the compound of claim 1; (iii) identifying the subject for administering the pharmaceutical composition comprising the compound of claim 1; (iv) assessing the subject for pre-administration vital signs and collecting biological samples from the subject to establish and record baseline physiological, metabolic profile, and medical history of the subject for pre-administration assessment; (v) administering a therapeutically effective amount of the pharmaceutical composition comprising the compound of claim 1 to the subject; (vi) repeating the administration in step (v) as per an established protocol for treating the disorder or disease in the subject; (vii) collecting biological samples and recording the vital signs from the subject to establish and record baseline physiological, metabolic profile at each step post-administration of step (v) and step (vi) as per the established protocol for post-administration assessment; (viii) comparing the results obtained from the pre-administration assessment and post-administration assessment to check for the potency and efficiency of the compound of claim 1 in preventing or treating the disorder or disease in the subject, wherein the disorder or disease is a signal transducer and activator of transcription 3 (STAT3) activation-dependent disorder or disease involving irregular activation of STAT3 protein, wherein the pre-administration and post-administration assessment include in-vivo measures based baseline assessment, and in-vitro assays conducted as biochemical and cellular assays, and cell-based assays on the biological samples obtained in steps (iv) and (vii), and wherein the compound of claim 1 is a small-molecule inhibitor targeting signal transducer and activator of transcription 3 (STAT3) as a potent and efficient selective inhibitor of STAT3 at a nanomolar concentration.

10. The method of claim 9, wherein the disorder or disease includes triple-negative breast cancer (TNBC), other cancers with STAT3 activation including prostate cancer, liver cancer, leukemia, lymphoma, multiple myeloma, colorectal cancer, lung cancer, and brain cancers including glioblastoma, and neuroblastoma, different metabolic diseases including Coronavirus disease 2019 (COVID-19), myalgic encephalomyelitis or chronic fatigue syndrome (ME/CFS).

11. The method of claim 9, wherein the compound targets suppression of phosphorylation at tyrosine 705 (Y705) of STAT3 protein.

12. The method of claim 9, wherein the baseline assessment includes comprehensive documentation of patient characteristics including demographic information comprising data on age, gender, ethnicity; social factors including support system, employment status, lifestyle habits; medical history including existing medical conditions and/or comorbidities, severity of the primary disease; clinical symptoms including pain level, functional ability, fatigue levels, mood; routine and disease or disorder specific laboratory test values including disease or disorder specific blood tests and imaging results indicating disease progression and grade; quality of life measures including self-reported assessments of physical and mental well-being; and psychological factors including anxiety levels, depression, coping mechanisms.

13. The method of claim 9, wherein the biochemical and cellular assays include protein thermal shift (PTS) assay, fluorescence polarization analyses, high-throughput STAT binding protein array assay, immunoblot assay, 6× luciferase reporter gene assay array for selective STAT3 selective binding and molecular interaction assessment at a nanomolar concentration of the compound of claim 1.

14. The method of claim 9, wherein the cell-based assays include assessment of specific STAT3 activation inhibition targeting suppression of phosphorylation at tyrosine 705 (Y705) of STAT3 protein, lactate dehydrogenase cell cytotoxicity assay, dose-response analysis for apoptosis in cell-based assays, annexin-V/propidium iodide dual labeling assay, TUNEL staining assay, and apoptotic gene array assay at a nanomolar concentration of the compound of claim 1.


SEQUENCE LISTING (137 sequences)

SEQ ID NO: 1 - DNA, 24 bp, synthetic construct
  source 1..24: mol_type = other DNA
gatccttctgggaattcctagatc

SEQ ID NO: 2 - DNA, 17 bp, Homo sapiens
  source 1..17: mol_type = genomic DNA
ttttgagggaaaatgat

SEQ ID NO: 3 - DNA, 15 bp, synthetic construct
  source 1..15: mol_type = other DNA
ttgaggtggaatgat

SEQ ID NO: 4 - DNA, 23 bp, synthetic construct
  source 1..23: mol_type = other DNA
agacctggtttggggatcttaat

SEQ ID NO: 5 - DNA, 20 bp, synthetic construct
  source 1..20: mol_type = other DNA
agcaatgagctggtctccca

SEQ ID NO: 6 - DNA, 20 bp, synthetic construct
  source 1..20: mol_type = other DNA
ggtactctgtgggttgctcc

SEQ ID NO: 7 - DNA, 20 bp, synthetic construct
  source 1..20: mol_type = other DNA
gtgagctgccaggatgaact

SEQ ID NO: 8 - DNA, 20 bp, synthetic construct
  source 1..20: mol_type = other DNA
tcaaagtcggacagcctcac

SEQ ID NO: 9 - DNA, 20 bp, synthetic construct
  source 1..20: mol_type = other DNA
atccattgattgccccagca

SEQ ID NO: 10 - DNA, 20 bp, synthetic construct
  source 1..20: mol_type = other DNA
gcctcttcacgggctagaaa

SEQ ID NO: 11 - DNA, 20 bp, synthetic construct
  source 1..20: mol_type = other DNA
taaagcgggactgaacaccc

SEQ ID NO: 12 - DNA, 20 bp, synthetic construct
  source 1..20: mol_type = other DNA
aaccagcgcatggacagtta

SEQ ID NO: 13 - DNA, 20 bp, synthetic construct
  source 1..20: mol_type = other DNA
gacttgaccaccgaacccat

SEQ ID NO: 14 - DNA, 20 bp, synthetic construct
  source 1..20: mol_type = other DNA
ggagcctaatcagcgaggtt

SEQ ID NO: 15 - DNA, 20 bp, synthetic construct
  source 1..20: mol_type = other DNA
gccagagacggcttctatca

SEQ ID NO: 16 - DNA, 20 bp, synthetic construct
  source 1..20: mol_type = other DNA
cccagtccacctcgatttcc

SEQ ID NO: 17 - DNA, 20 bp, synthetic construct
  source 1..20: mol_type = other DNA
aaacggtccttacccgtgac

SEQ ID NO: 18 - DNA, 20 bp, synthetic construct
  source 1..20: mol_type = other DNA
gggggaaaagcgatcctgaa

SEQ ID NO: 19 - DNA, 20 bp, synthetic construct
  source 1..20: mol_type = other DNA
ctgtgtcatcctcccagcag

SEQ ID NO: 20 - DNA, 20 bp, synthetic construct
  source 1..20: mol_type = other DNA
agcactgtccaacaacacca

SEQ ID NO: 21 - DNA, 20 bp, synthetic construct
  source 1..20: mol_type = other DNA
cttcaggtggctggagtgag

SEQ ID NO: 22 - DNA, 20 bp, synthetic construct
  source 1..20: mol_type = other DNA
cttaaggcgcgaaatctgcc

SEQ ID NO: 23 - DNA, 20 bp, synthetic construct
  source 1..20: mol_type = other DNA
gcgatctcggagaagtccag

SEQ ID NO: 24 - DNA, 20 bp, synthetic construct
  source 1..20: mol_type = other DNA
gaactgggggaggattgtgg

SEQ ID NO: 25 - DNA, 20 bp, synthetic construct
  source 1..20: mol_type = other DNA
ttgtggctcagataggcacc

SEQ ID NO: 26 - DNA, 23 bp, synthetic construct
  source 1..23: mol_type = other DNA
gtgtcaagactctcagacgagat

SEQ ID NO: 27 - DNA, 20 bp, synthetic construct
  source 1..20: mol_type = other DNA
agagggcatagttggggact

SEQ ID NO: 28 - DNA, 19 bp, synthetic construct
  source 1..19: mol_type = other DNA
tgtctcagagcaaccggga

SEQ ID NO: 29 - DNA, 21 bp, synthetic construct
  source 1..21: mol_type = other DNA
catccaaactgctgctgtgcg

SEQ ID NO: 30 - DNA, 20 bp, synthetic construct
  source 1..20: mol_type = other DNA
cctgctttgtccccatcgag

SEQ ID NO: 31 - DNA, 19 bp, synthetic construct
  source 1..19: mol_type = other DNA
gaggctggccagagaagac

SEQ ID NO: 32 - DNA, 20 bp, synthetic construct
  source 1..20: mol_type = other DNA
cggaggcatcaacccagatt

SEQ ID NO: 33 - DNA, 21 bp, synthetic construct
  source 1..21: mol_type = other DNA
gatggtggtgtagaccttccg

SEQ ID NO: 34 - DNA, 19 bp, synthetic construct
  source 1..19: mol_type = other DNA
gaccaacacccttcccacc

SEQ ID NO: 35 - DNA, 20 bp, synthetic construct
  source 1..20: mol_type = other DNA
ttctgagccaatcaccgagc

SEQ ID NO: 36 - DNA, 21 bp, synthetic construct
  source 1..21: mol_type = other DNA
ggcagagaccatccaagaagt

SEQ ID NO: 37 - DNA, 20 bp, synthetic construct
  source 1..20: mol_type = other DNA
acacagcgcacatagagctt

SEQ ID NO: 38 - DNA, 20 bp, synthetic construct
  source 1..20: mol_type = other DNA
ggccaactcagcttttgtgg

SEQ ID NO: 39 - DNA, 20 bp, synthetic construct
  source 1..20: mol_type = other DNA
catggtggagttgtcccgaa

SEQ ID NO: 40 - DNA, 20 bp, synthetic construct
  source 1..20: mol_type = other DNA
ctgattggacaggcatgggt

SEQ ID NO: 41 - DNA, 20 bp, synthetic construct
  source 1..20: mol_type = other DNA
gtgcctggaagtcaacggta

SEQ ID NO: 42 - DNA, 21 bp, synthetic construct
  source 1..21: mol_type = other DNA
tgcaaaagaaggcatgcacag

SEQ ID NO: 43 - DNA, 20 bp, synthetic construct
  source 1..20: mol_type = other DNA
tcttcaggttctcccccagg

SEQ ID NO: 44 - DNA, 20 bp, synthetic construct
  source 1..20: mol_type = other DNA
cccaaggactcagggacaac

SEQ ID NO: 45 - DNA, 20 bp, synthetic construct
  source 1..20: mol_type = other DNA
tggaggctgcgaaggatttt

SEQ ID NO: 46 - DNA, 20 bp, synthetic construct
  source 1..20: mol_type = other DNA
cggatctcttcctgctcgac

SEQ ID NO: 47 - DNA, 20 bp, synthetic construct
  source 1..20: mol_type = other DNA
cacagtagtaggcggcgtag

SEQ ID NO: 48 - DNA, 20 bp, synthetic construct
  source 1..20: mol_type = other DNA
atctacggtttgctgtgggg

SEQ ID NO: 49 - DNA, 20 bp, synthetic construct
  source 1..20: mol_type = other DNA
aaaggtgtcaggcagaaggg

SEQ ID NO: 50 - DNA, 19 bp, synthetic construct
  source 1..19: mol_type = other DNA
ctagggcactggcgaaacc

SEQ ID NO: 51 - DNA, 20 bp, synthetic construct
  source 1..20: mol_type = other DNA
actgaaacgctctgatggca

SEQ ID NO: 52 - DNA, 20 bp, synthetic construct
  source 1..20: mol_type = other DNA
ccctgtgtcttccccttcac

SEQ ID NO: 53 - DNA, 20 bp, synthetic construct
  source 1..20: mol_type = other DNA
gtcaggagaggccccataga

SEQ ID NO: 54 - DNA, 20 bp, synthetic construct
  source 1..20: mol_type = other DNA
ggtgtggagttcctgacgtt

SEQ ID NO: 55 - DNA, 20 bp, synthetic construct
  source 1..20: mol_type = other DNA
gcataggcatgggccaaaac

SEQ ID NO: 56 - DNA, 20 bp, synthetic construct
  source 1..20: mol_type = other DNA
gcaatgctgatgggaaaccc

SEQ ID NO: 57 - DNA, 20 bp, synthetic construct
  source 1..20: mol_type = other DNA
agaagccccacttcttgtcg

SEQ ID NO: 58 - DNA, 20 bp, synthetic construct
  source 1..20: mol_type = other DNA
ctctttccgtccttcccgtc

SEQ ID NO: 59 - DNA, 20 bp, synthetic construct
  source 1..20: mol_type = other DNA
ctcagccccaagacacacac

SEQ ID NO: 60 - DNA, 20 bp, synthetic construct
  source 1..20: mol_type = other DNA
ctctggcacgtcttgacctt

SEQ ID NO: 61 - DNA, 20 bp, synthetic construct
  source 1..20: mol_type = other DNA
accattcttctgcctcctgc

SEQ ID NO: 62 - DNA, 20 bp, synthetic construct
  source 1..20: mol_type = other DNA
gcccctcatccttccagaac

SEQ ID NO: 63 - DNA, 20 bp, synthetic construct
  source 1..20: mol_type = other DNA
ctgttgctgagtgtccgagt

SEQ ID NO: 64 - DNA, 20 bp, synthetic construct
  source 1..20: mol_type = other DNA
acaacaggcccaaaaaggga

SEQ ID NO: 65 - DNA, 20 bp, synthetic construct
  source 1..20: mol_type = other DNA
aggtaccatggccccaaatg

SEQ ID NO: 66 - DNA, 19 bp, synthetic construct
  source 1..19: mol_type = other DNA
ccatggtgcccctaggtct

SEQ ID NO: 67 - DNA, 20 bp, synthetic construct
  source 1..20: mol_type = other DNA
caccactcggacgaggtaac

SEQ ID NO: 68 - DNA, 20 bp, synthetic construct
  source 1..20: mol_type = other DNA
ggggtcccacgaatcttgtt

SEQ ID NO: 69 - DNA, 21 bp, synthetic construct
  source 1..21: mol_type = other DNA
gtctagccaagatggcagtca

SEQ ID NO: 70 - DNA, 20 bp, synthetic construct
  source 1..20: mol_type = other DNA
tccttggcgaggcttttgat

SEQ ID NO: 71 - DNA, 20 bp, synthetic construct
  source 1..20: mol_type = other DNA
ccactcctgccccattcatt

SEQ ID NO: 72 - DNA, 20 bp, synthetic construct
  source 1..20: mol_type = other DNA
ttctgggctgttctcgcttc

SEQ ID NO: 73 - DNA, 20 bp, synthetic construct
  source 1..20: mol_type = other DNA
acgaccgcttaccttggcat

SEQ ID NO: 74 - DNA, 20 bp, synthetic construct
  source 1..20: mol_type = other DNA
aacaggacctgggcagaaag

SEQ ID NO: 75 - DNA, 20 bp, synthetic construct
  source 1..20: mol_type = other DNA
agcttcactgggtaacagca

SEQ ID NO: 76 - DNA, 22 bp, synthetic construct
  source 1..22: mol_type = other DNA
tgtctttgagaaagtacggcac

SEQ ID NO: 77 - DNA, 21 bp, synthetic construct
  source 1..21: mol_type = other DNA
aggtgagatggaaaagctggt

SEQ ID NO: 78 - DNA, 20 bp, synthetic construct
  source 1..20: mol_type = other DNA
ttggggatcctcggagatgt

SEQ ID NO: 79 - DNA, 20 bp, synthetic construct
  source 1..20: mol_type = other DNA
acagtgacagcctctgaacg

SEQ ID NO: 80 - DNA, 20 bp, synthetic construct
  source 1..20: mol_type = other DNA
ttcatcacggaaaggggagc

SEQ ID NO: 81 - DNA, 20 bp, synthetic construct
  source 1..20: mol_type = other DNA
ggatccactctggagggcta

SEQ ID NO: 82 - DNA, 20 bp, synthetic construct
  source 1..20: mol_type = other DNA
accgggagctcagattcttc

SEQ ID NO: 83 - DNA, 20 bp, synthetic construct
  source 1..20: mol_type = other DNA
gcctggttaactgggcttca

SEQ ID NO: 84 - DNA, 20 bp, synthetic construct
  source 1..20: mol_type = other DNA
tggcttctcccagcctatct

SEQ ID NO: 85 - DNA, 20 bp, synthetic construct
  source 1..20: mol_type = other DNA
tgtgatgcttgcaaagtgcc

SEQ ID NO: 86 - DNA, 20 bp, synthetic construct
  source 1..20: mol_type = other DNA
tgcttacagctaccacagcc

SEQ ID NO: 87 - DNA, 20 bp, synthetic construct
  source 1..20: mol_type = other DNA
ccagactgggcagagaaagg

SEQ ID NO: 88 - DNA, 20 bp, synthetic construct
  source 1..20: mol_type = other DNA
gcagccgccacattcaacag

SEQ ID NO: 89 - DNA, 19 bp, synthetic construct
  source 1..19: mol_type = other DNA
atttgggggaacgaagccg

SEQ ID NO: 90 - DNA, 20 bp, synthetic construct
  source 1..20: mol_type = other DNA
gtgttccaggggaagcacat

SEQ ID NO: 91 - DNA, 20 bp, synthetic construct
  source 1..20: mol_type = other DNA
gctcttgccagttccctcat

SEQ ID NO: 92 - DNA, 20 bp, synthetic construct
  source 1..20: mol_type = other DNA
caggccacatttcatgcacc

SEQ ID NO: 93 - DNA, 20 bp, synthetic construct
  source 1..20: mol_type = other DNA
ggaagtcctctggctcttcg

SEQ ID NO: 94 - DNA, 21 bp, synthetic construct
  source 1..21: mol_type = other DNA
tgatgctctaagtccactgcc

SEQ ID NO: 95 - DNA, 20 bp, synthetic construct
  source 1..20: mol_type = other DNA
ttgggaaacgcttacctggg

SEQ ID NO: 96 - DNA, 20 bp, synthetic construct
  source 1..20: mol_type = other DNA
aggtcagttccttgtggagc

SEQ ID NO: 97 - DNA, 20 bp, synthetic construct
  source 1..20: mol_type = other DNA
gaaggtagagcttgggcagg

SEQ ID NO: 98 - DNA, 20 bp, synthetic construct
  source 1..20: mol_type = other DNA
acacagagggagactggtgg

SEQ ID NO: 99 - DNA, 20 bp, synthetic construct
  source 1..20: mol_type = other DNA
ttggcgttgtcgaagtcaga

SEQ ID NO: 100 - DNA, 20 bp, synthetic construct
  source 1..20: mol_type = other DNA
cgggatatgccagatgggac

SEQ ID NO: 101 - DNA, 20 bp, synthetic construct
  source 1..20: mol_type = other DNA
tggacactgggtacatcagc

SEQ ID NO: 102 - DNA, 21 bp, synthetic construct
  source 1..21: mol_type = other DNA
tgcgatggagtcagaggaaac

SEQ ID NO: 103 - DNA, 20 bp, synthetic construct
  source 1..20: mol_type = other DNA
ggctagcgctaagaagcaga

SEQ ID NO: 104 - DNA, 20 bp, synthetic construct
  source 1..20: mol_type = other DNA
aaaacaaccctcagacgcca

SEQ ID NO: 105 - DNA, 20 bp, synthetic construct
  source 1..20: mol_type = other DNA
ggagagtggatgaaggctgg

SEQ ID NO: 106 - DNA, 20 bp, synthetic construct
  source 1..20: mol_type = other DNA
cagctctgcatcgttttggg

SEQ ID NO: 107 - DNA, 20 bp, synthetic construct
  source 1..20: mol_type = other DNA
ctgttttagctgctggcgac

SEQ ID NO: 108 - DNA, 20 bp, synthetic construct
  source 1..20: mol_type = other DNA
tcattgctactgccctctgc

SEQ ID NO: 109 - DNA, 20 bp, synthetic construct
  source 1..20: mol_type = other DNA
tcttctctgggttggcacac

SEQ ID NO: 110 - DNA, 21 bp, synthetic construct
  source 1..21: mol_type = other DNA
acttgagtgagtccaagctgt

SEQ ID NO: 111 - DNA, 20 bp, synthetic construct
  source 1..20: mol_type = other DNA
agccagctgctgtcatcttt

SEQ ID NO: 112 - DNA, 20 bp, synthetic construct
  source 1..20: mol_type = other DNA
cacaaggtggcaggatgtct

SEQ ID NO: 113 - DNA, 20 bp, synthetic construct
  source 1..20: mol_type = other DNA
tccccgactgagcctgatta

SEQ ID NO: 114 - DNA, 17 bp, synthetic construct
  source 1..17: mol_type = other DNA
ctcacgggagctgctgg

SEQ ID NO: 115 - DNA, 20 bp, synthetic construct
  source 1..20: mol_type = other DNA
ggttcctgttgctgctgttg

SEQ ID NO: 116 - DNA, 21 bp, synthetic construct
  source 1..21: mol_type = other DNA
ccccatactcctccccatcat

SEQ ID NO: 117 - DNA, 20 bp, synthetic construct
  source 1..20: mol_type = other DNA
ctgcctccaggaacaacaga

SEQ ID NO: 118 - DNA, 20 bp, synthetic construct
  source 1..20: mol_type = other DNA
tccaccaacctcatgtcagc

SEQ ID NO: 119 - DNA, 20 bp, synthetic construct
  source 1..20: mol_type = other DNA
gccatgggacttgagcatct

SEQ ID NO: 120 - DNA, 21 bp, synthetic construct
  source 1..21: mol_type = other DNA
acctcgcactctcagtttcac

SEQ ID NO: 121 - DNA, 20 bp, synthetic construct
  source 1..20: mol_type = other DNA
tacttgccgctgtcgatctg

SEQ ID NO: 122 - DNA, 20 bp, synthetic construct
  source 1..20: mol_type = other DNA
ggtcaaggacctccagcatc

SEQ ID NO: 123 - DNA, 20 bp, synthetic construct
  source 1..20: mol_type = other DNA
agaatcttgcactgggaggc

SEQ ID NO: 124 - DNA, 20 bp, synthetic construct
  source 1..20: mol_type = other DNA
gcctggctctgaagtggaat

SEQ ID NO: 125 - DNA, 20 bp, synthetic construct
  source 1..20: mol_type = other DNA
ccaaagccgagctgttgttt

SEQ ID NO: 126 - DNA, 20 bp, synthetic construct
  source 1..20: mol_type = other DNA
cccctagactcaggcttcct

SEQ ID NO: 127 - DNA, 20 bp, synthetic construct
  source 1..20: mol_type = other DNA
agttcgttctgccccatcag

SEQ ID NO: 128 - DNA, 20 bp, synthetic construct
  source 1..20: mol_type = other DNA
gaaaaccagcgcgtttccat

SEQ ID NO: 129 - DNA, 20 bp, synthetic construct
  source 1..20: mol_type = other DNA
gggaattgggggtcagacag

SEQ ID NO: 130 - DNA, 20 bp, synthetic construct
  source 1..20: mol_type = other DNA
atcctgttgttgctgtggct

SEQ ID NO: 131 - DNA, 20 bp, synthetic construct
  source 1..20: mol_type = other DNA
gggtgtagacgaaccggatg

SEQ ID NO: 132 - DNA, 20 bp, synthetic construct
  source 1..20: mol_type = other DNA
ggctcggtcctgtatgtgtc

SEQ ID NO: 133 - DNA, 20 bp, synthetic construct
  source 1..20: mol_type = other DNA
atcggtccttgtatgggcag

SEQ ID NO: 134 - DNA, 20 bp, synthetic construct
  source 1..20: mol_type = other DNA
gcattcagatcccgaaacgc

SEQ ID NO: 135 - DNA, 20 bp, synthetic construct
  source 1..20: mol_type = other DNA
gccctcgaaggtgagttgaa

SEQ ID NO: 136 - DNA, 20 bp, synthetic construct
  source 1..20: mol_type = other DNA
agaatgaggaggagaccccc

SEQ ID NO: 137 - DNA, 20 bp, synthetic construct
  source 1..20: mol_type = other DNA
ttcctttgggggaagatgcc